SEQUENCE LISTING (16 sequences)

SEQ ID NO: 1 - protein, 5 aa, Artificial Sequence
  CDR sequence
SSWMN

SEQ ID NO: 2 - protein, 17 aa, Artificial Sequence
  CDR sequence
RIYPGDGDTNYNVKFKG

SEQ ID NO: 3 - protein, 12 aa, Artificial Sequence
  CDR sequence
SGFITTVRDFDY

SEQ ID NO: 4 - protein, 15 aa, Artificial Sequence
  CDR sequence
RASESVDTFGISFMN

SEQ ID NO: 5 - protein, 7 aa, Artificial Sequence
  CDR sequence
EASNQGS

SEQ ID NO: 6 - protein, 9 aa, Artificial Sequence
  CDR sequence
QQSKEVPFT

SEQ ID NO: 7 - protein, 121 aa, Artificial Sequence
  VH sequence
EVQLVESGGGLVQPGGSLRLSCAASGFTFSSSWMNWVRQAPGKGLEWVGRIYPGDGDTNYNVKFKGRFTISRDDSKNSLYLQMNSLKTEDTAVYYCARSGFITTVRDFDYWGQGTLVTVSS

SEQ ID NO: 8 - protein, 111 aa, Artificial Sequence
  VL sequence
EIVLTQSPDFQSVTPKEKVTITCRASESVDTFGISFMNWFQQKPDQSPKLLIHEASNQGSGVPSRFSGSGSGTDFTLTINSLEAEDAATYYCQQSKEVPFTFGGGTKVEIK

SEQ ID NO: 9 - protein, 5 aa, Artificial Sequence
  CDR sequence
SSWMN

SEQ ID NO: 10 - protein, 17 aa, Artificial Sequence
  CDR sequence
RIYPGDGDTNYNGKFKG

SEQ ID NO: 11 - protein, 12 aa, Artificial Sequence
  CDR sequence
SGFITTVLDFDY

SEQ ID NO: 12 - protein, 15 aa, Artificial Sequence
  CDR sequence
RASESVDTFGISFMN

SEQ ID NO: 13 - protein, 7 aa, Artificial Sequence
  CDR sequence
AASNQGS

SEQ ID NO: 14 - protein, 9 aa, Artificial Sequence
  CDR sequence
QQSKEVPFT

SEQ ID NO: 15 - protein, 121 aa, Artificial Sequence
  VH sequence
EVQLVESGGGLVQPGGSLRLSCAASGFTFSSSWMNWVRQAPGKGLEWVGRIYPGDGDTNYNGKFKGRFTISRDDSKNSLYLQMNSLKTEDTAVYYCARSGFITTVLDFDYWGQGTLVTVSS

SEQ ID NO: 16 - protein, 111 aa, Artificial Sequence
  VL sequence
EIVLTQSPDFQSVTPKEKVTITCRASESVDTFGISFMNWFQQKPDQSPKLLIHAASNQGSGVPSRFSGSGSGTDFTLTINSLEAEDAATYYCQQSKEVPFTFGGGTKVEIK

(19) United States
(12) Patent Application Publication
(10) Pub. No.: US 2015/0044168 A1
(43) Pub. Date: Feb. 12, 2015

(54) Treatment of Multiple Sclerosis With Anti-CD19 Antibody

(71) Applicant: Medlmmune, LLC, Gaithersburg, MD (US)

(72) Inventors: Ronald Herbst, Gaithersburg, MD (US); Volker Armin Knappertz, Potomac, MD (US); Laura Lee Carter, Ann Arbor, MI (US); Yue Wang, Gaithersburg, MD (US)

(21) Appl. No.: 14/384,714

(22) PCT Filed: Mar. 11, 2013
(86) PCT No.: PCT/US13/30247

§ 371 Date: Sep. 12, 2014

Related U.S. Application Data
(60) Provisional application No. 61/609,704, filed on Mar. 12, 2012.

(51) Int. Cl. C07K 16/28; A61K 45/06; A61K 39/395

(52) U.S. Cl. CPC C07K 16/2803; A61K 39/39533; A61K 45/06
USPC 424/85.6; 424/133.1; 424/144.1; 424/181.1

(57) ABSTRACT

The present invention provides for the treatment of multiple sclerosis through the use of chimeric and humanized versions of anti-CD19 antibodies that may mediate ADCC, CDC, and/or apoptosis.

### BACKGROUND

Multiple sclerosis (“MS”) is a chronic inflammatory disease of the central nervous system. The characteristic pathological feature, and the feature still used as the primary basis for diagnosis of MS, is demyelination of the myelin sheath of neurons in the central nervous system. MS affects as many as 400,000 people in the United States, and approximately 1 million people worldwide. Typically, MS begins as a relapsing-remitting disease (RRMS) with periodic episodes of associated symptoms (e.g. various forms of neuritis). Often RRMS eventually changes to a progressive course of disease, secondary progressive MS (SPMS), characterized by more CNS tissue damage which results in more debilitating symptoms. However, in 10 to 20% of individuals, the disease initially develops in a progressive form known as primary progressive MS (PPMS). In addition, there is a rarer form of the disease, progressive-relapsing (PR) MS.

Current hypotheses favour the concept that T cells play a pivotal role in the pathogenesis of Multiple Sclerosis (MS), which was initially based upon the observation that T cells are the predominant lymphocyte class present in MS lesions (Windhagen, et al., Cytokine, secretion of myelin basic protein reactive T cells in patients with multiple sclerosis. Journal of Neuroimmunology, 91:1-9, 1998; Hafler, D. A., et al., Oral administration of myelin induces antigen-specific TGF-beta 1 secreting cells in patients with multiple sclerosis Annals of the New York Academy of Science, 835:120-131, 1997; Lovett-Racke, A. E., et al., Decreased dependence of myelin basic protein-reactive T cells on CD28-mediated co-stimulation in multiple sclerosis patients, Journal of Clinical Investigation, 101:725-730, 1998). This continues to be a cardinal hallmark of the disease, and is supported by a number of observations. For example, active CD4+T helper cells bearing anti-myelin T Cell Receptors (TCRs) are present in the cerebrospinal fluid (CSF) of patients with MS. In addition, elevated levels of Th1-like cytokines have been detected in the CSF of patients with MS and have been correlated with worsening of the disease in some cases (Calabresi et al, Cytokine expression in cells derived from CSF of multiple sclerosis patients. Journal of Neuroimmunology, 89:198-205, 1998).

There is evidence that B cells may be involved in the development and perpetuation of the MS disease process including: (1) elevated immunoglobulin levels in the CSF of MS patients (Link, H., et al., Immunoglobulins in multiple sclerosis and infections of the nervous system, Archives of Neurology, 25:326-344, 1971; Link, H., et al., Immunoglobulin class and light chain type of oligoclonal bands in CSF in multiple sclerosis determined by agarose gel electrophoresis and immunofixation. Ann Neurol, 6(2):107-110, 1979; Perez, L, et al., B cells capable of spontaneous IgG secretion in cerebrospinal fluid from patients with multiple sclerosis: dependency on local IL-6 production. Clinical Experimental Immunology, 101:449-452, 1995), (2) oligoclonal banding in the CSF of MS patients (Link, H., et al., Immunoglobulin class and light chain type of oligoclonal bands in CSF in multiple sclerosis determined by agarose gel electrophoresis and immunofixation. Ann Neurol, 6(2):107-110, 1979), (3) the presence of anti-myelin antibodies in the CSF of MS patients (Sun, J. H., et al, B cell responses to myelin-oligodendrocyte glycoprotein in multiple sclerosis Journal of Immunology, 146:1490-1495, 1991), (4) the demonstration that antibodies from the CSF of MS patients may contribute to the overall extent of tissue injury in these patients (Lassmann, H., et al., Experimental allergic encephalomyelitis: the balance between encephalitogenic T lymphocytes and demyelinating antibodies determines size and structure of demyelinated lesions. Acta Neuropathology, 75:566-576, 1988), and (5) the presence of CD19+B cells and CD19+138+ plasma blasts in CSF of MS patients (Winges, K M et al., Analysis of multiple sclerosis cerebrospinal fluid reveals a continuum of clonally related antibody-secreting cells that are predominantly plasma blasts. Journal of Neuroimmunology, 192:226-234, 2007, Cepok S et al. (2005). Brain 128(Pt 7):1667-76).

Therefore, a need exists for methods which may be used to therapeutically treat MS by targeting B cells, including plasmablasts/plasma cells, in an individual; particularly in an individual who has the disease condition.

### SUMMARY

In one aspect, the disclosure provides methods of treating multiple sclerosis, comprising administering to a subject in need thereof a therapeutically-effective amount of an antibody, wherein the antibody is a humanized antibody or antigen binding fragment thereof, that binds a CD19 antigen.

In certain embodiments, the multiple sclerosis disease is selected from the group consisting of relapsing-remitting (RR) MS, primary-progressive (PP) MS, secondary-progressive (SP) MS, relapsing-progressive (RP) MS and progressive-relapsing (PR) MS. In an embodiment the multiple sclerosis disease is RRMS. In an alternative embodiment the multiple sclerosis disease is a progressive form of MS selected from PPMS, SPMS, and PR MS.

In certain embodiments, the antibody comprises a VH and a VL, wherein the VH comprises a VH CDR1 having an amino acid sequence of SEQ ID NO: 1, a VH CDR2 having an amino acid of SEQ ID NO: 2 and VH CDR3 having an amino acid sequence of SEQ ID NO: 3.

In certain embodiments, the antibody comprises a VH and a VL, wherein the VL comprises a VL CDR1 having an amino acid sequence of SEQ ID NO: 4, a VL CDR2 having an amino acid of SEQ ID NO: 5 and VL CDR3 having an amino acid sequence of SEQ ID NO: 6.

In other embodiments, the VH comprises an amino acid sequence of SEQ ID NO: 7. In certain embodiments, the VL comprises an amino acid sequence of SEQ ID NO: 8.

In alternative embodiments, the antibody comprises an Fc variant, wherein the Fc variant has an altered affinity for one or more Fc ligands selected from the group consisting of: C1q, FcγRI, FcγRIIA, FcγRIIB and FcγRIIIA. In certain embodiments, the Fc variant has an affinity for the Fc receptor FcγRIIIA that is at least about 5 fold lower than that of a comparable molecule, and wherein said Fc variant has an affinity for the Fc receptor FcγRIIB that is within about 2 fold of that of a corresponding non-variant Fc molecule.

In some embodiments, the antibody has an enhanced ADCC activity.

In certain embodiments, the method of treating multiple sclerosis comprises depletion of B cells selected from the group consisting of: circulating B cells, blood B cells, splenic B cells, marginal zone B cells, follicular B cells, peritoneal B cells and bone marrow B cells. In certain embodiments, the method comprises depletion of B cells selected from the group consisting of: progenitor B cells, early pro-B cells, late pro-B cells, large-pre-B cells, small pre-B cells, immature B cells, mature B cells, antigen stimulated B cells, plasmablasts and plasma cells.

In some embodiments, the depletion reduces B cell levels by at least about 20%, at least about 30%, at least about 40%, at least about 50%, at least about 60%, at least about 70%, at least about 80%, at least about 90%, at least about 95%, or about 100%.

In other embodiments, the depletion persists for a time period selected from the group consisting of: at least 1 week, at least 2 weeks, at least 3 weeks, at least 4 weeks, at least 5 weeks, at least 6 weeks, at least 7 weeks, at least 8 weeks, at least 3 months, at least 4 months, at least 5 months, at least 6 months, at least 7 months, at least 8 months, at least 9 months, at least 10 months, at least 11 months or at least 12 months.

In some embodiments, the antibody is conjugated to a cytotoxic agent. In some embodiments, the antibody is co-administered with an anti-CD20, anti-CD52, or anti-CD22 antibody. In some embodiments, the antibody is co-administered with an interferon-beta, Copaxone™, corticosteroids, cyclosporine, calcineurin inhibitors, azathioprine, Rapamune™, Cellcept™, methotrexate or mitoxantrone

The disclosure also provides methods of treating multiple sclerosis in a human, comprising administering to a patient in need thereof a composition comprising a plurality of monoclonal antibodies that bind a CD19 antigen, wherein 80-100% of the antibodies are afucosylated.

In some embodiments, the antibody comprises a VH and a VL, wherein the VH comprises a VH CDR1 having an amino acid sequence of SEQ ID NO: 1, a VH CDR2 having an amino acid of SEQ ID NO: 2 and VH CDR3 having an amino acid sequence of SEQ ID NO: 3.

In other embodiments, the antibody comprises a VH and a VL, wherein the VL comprises a VL CDR1 having an amino acid sequence of SEQ ID NO: 4, a VL CDR2 having an amino acid of SEQ ID NO: 5 and VL CDR3 having an amino acid sequence of SEQ ID NO: 6.

In alternative embodiments, the VH comprises an amino acid sequence of SEQ ID NO: 7. In some embodiments, the VL comprises an amino acid sequence of SEQ ID NO: 8.

The disclosure contemplates all combinations of any of the foregoing aspects and embodiments, as well as combinations with any of the embodiments set forth in the detailed description and examples.

### BRIEF DESCRIPTION OF THE DRAWINGS

FIG. 1: Expression of CD19 on plasma cells from human CD19 transgenic (huCD19Tg) mice

FIG. 2: Effect of 16C4-aFuc on antibody titres and plasma cell numbers in ovalbumin (ova) immunized human CD 19 transgenic (huCD19Tg) mice

FIG. 3: Treatment of SLE1xhuCD19Tg mice with 16C4-aFuc—effect on blood and tissue B cells

FIG. 4: Treatment of SLE mice with 16C4-aFuc—effect on spleen and bone marrow plasma cells

FIG. 5: Treatment of SLE mice with 16C4-aFuc—effect on anti-dsDNA autoantibodies

FIG. 6: Treatment of SLE mice with 16C4-aFuc—effect on serum immunoglobulins over time

FIG. 7: Treatment of SLE1xhuCD19Tg mice with 16C4-aFuc—reduction of autoantibodies in serum

FIG. 8: CD27+CD38high cells positive for CD19 have a plasma cell phenotype and secrete IgG (8A FACS panels; 8B morphology; 8C Total IgG ELISpot)

FIG. 9: CD 19 (left column) and CD20 (right column) surface expression levels in various human tissues.

FIG. 10: A CD19 negative ASC population from BM contains most of the humoral memory to vaccine antigens.

FIG. 11: Inhibition of the Plasma Cell Signature in Whole Blood Following 16C4afucTreatment (Up to Day 85). Transcript levels of the PC signature were evaluated in WB from scleroderma patients by whole genome array on days 3, 29, and 85 following treatment with 16C4afuc or placebo. Median fold change values compared to baseline in WB are shown for all patients at each timepoint evaluated. Error bars are median absolute deviation. * indicates statistically significant differences between baseline and post-administration values (p<0.01; Mann-Whitney U test).

FIG. 12: Inhibition of the Plasma Cell Signature in Skin Following 16C4afuc Treatment. Transcript levels of the PC signature in skin were evaluated by TaqMan qPCR prior to therapy and on day 29 post 16C4afuc or placebo treatment. Fold change values were calculated using expression levels of housekeeping genes, then by comparison to each patient's baseline expression of the PC signature. Dotted line represents the baseline fold change value (set to 1). Black bars represent median fold change values. * indicates statistically significant differences between baseline and post-treatment values (p<0.05).

FIG. 13: Concordant Inhibition of the Plasma Cell Signature between Blood and Skin from CP200 Patients. Median fold change values for the PC signature at day 29 post 16C4afuc treatment in both blood and skin were calculated as described. Shown is the correlation scatter plot of the inhibition of the PC signature between skin (y axis) and whole blood (x axis) in subjects with scleroderma enrolled in CP200. r=Spearman rank correlation coefficient. p<0.05 indicates a significant correlation.

FIG. 14: 16C4afuc dependent killing of in vitro differentiated PC. A. Representative data illustrating the relative lack or sufficiency of PC (CD27 high CD38 high) under non-differentiating or PC differentiating conditions after 6.5 days in culture. B. CD19 and CD20 expression of in vitro differentiated PC after 6.5 days in culture. C. Average number of viable plasma cells per well post ADCC assay. Each individual donor is graphed and the mean (±S.D. n=6 replicates for test conditions and n=10 replicates for no antibody controls) is shown for each group. *** indicates a p-value≦0.001 in a pairwise comparison with no antibody controls.

FIG. 15: 16C4afuc dependent killing of human plasma cells freshly isolated from same day shipped bone marrow. A. Representative data illustrating the identification of PC (CD27 high CD38 high) following B cell enrichment. B. CD19 and CD20 expression of the PC from each donor. C. Average number of viable plasma cells per well post-ADCC assay. Each individual donor is graphed and the mean (±S.D. n=6 replicates) is shown for each group. *** indicates a p-value≦0.001 in a pairwise comparison with no antibody controls.

FIG. 16: The results showed that CD19+CD20− plasmablasts and plasma cells are enriched in the CSF of RRMS patients.

FIG. 17 shows 3 representative flow cytometry plots

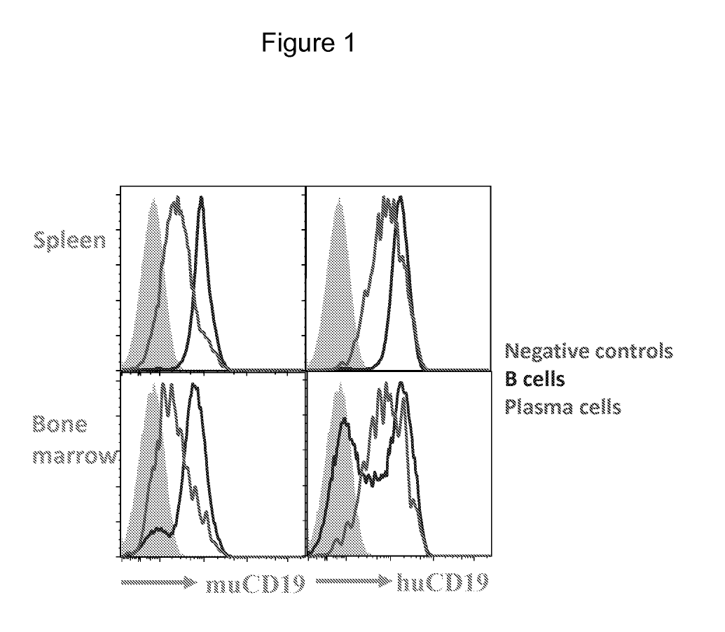

### DETAILED DESCRIPTION

The present disclosure relates to human, humanized, or chimeric anti-CD 19 antibodies that bind to the human CD19 antigen. The present disclosure is also directed to compositions comprising human, humanized, or chimeric anti-CD 19 antibodies that may mediate one or more of the following: complement-dependent cell-mediated cytotoxicity (CDC), antigen-dependent cell-mediated-cytotoxicity (ADCC), and programmed cell death (apoptosis). The present disclosure is also directed to compositions comprising human, humanized, or chimeric anti-CD19 antibodies of the IgG1 and/or IgG3 human isotype, as well as to compositions comprising human, humanized, or chimeric anti-CD19 antibodies of the IgG2 and/or IgG4 human isotype that may mediate human ADCC, CDC, or apoptosis. The present disclosure further relates to methods of using human, humanized, or chimeric anti-CD19 antibodies for the treatment of MS.

“Multiple sclerosis” refers to the chronic and often disabling disease of the central nervous system characterized by the progressive destruction of the myelin. As discussed above, there are four internationally recognized forms of MS, namely, primary progressive multiple sclerosis (PPMS), relapsing-remitting multiple sclerosis (RRMS), secondary progressive multiple sclerosis (SPMS), and progressive relapsing multiple sclerosis (PRMS).

“Relapsing-remitting multiple sclerosis” or “RRMS” is characterized by clearly defined disease relapses (also known as exacerbations) with full recovery or with sequelae and residual deficit upon recovery periods between disease relapses characterized by a lack of disease progression. The defining elements of RRMS are episodes of acute worsening of neurologic function followed by a variable degree of recovery, with a stable course between attacks (Lublin, F. D. & Reingold, S. O (1996) Neurology (46) 907-911). Relapses can last for days, weeks or months and recovery can be slow and gradual or almost instantaneous. The vast majority of people presenting with MS are first diagnosed with RRMS. This is typically when they are in their twenties or thirties, though diagnoses occurring much earlier or later are known. Twice as many women as men present with this sub-type of MS. During relapses, myelin, a protective insulating sheath around the nerve fibres (neurons) in the white matter regions of the central nervous system (CNS), may be damaged in an inflammatory response by the body's own immune system. This causes a wide variety of neurological symptoms that vary considerably depending on which areas of the CNS are damaged. Immediately after a relapse, the inflammatory response dies down and a special type of glial cell in the CNS (called an oligodendrocyte) sponsors remyelination—a process whereby the myelin sheath around the axon may be repaired. It is this remyelination that may be responsible for the remission. Approximately 50% of patients with RRMS convert to SPMS within 10 years of disease onset. After 30 years, this figure rises to 90%. At any one time, the relapsing-remitting form of the disease accounts around 55% of all people with MS.

Primary progressive multiple sclerosis or PPMS is characterized by disease progression with unrelenting deterioration of neurological function from the onset allowing for occasional plateauing and at times minor improvements in neurological functioning. The essential element in PPMS is a gradual and almost continuously worsening function allowing for minor fluctuations but without distinct relapses (Lublin, F. D. & Reingold, S. O (1996)). PPMS differs from RRMS and SPMS in that onset is on average about 10 years later than RRMS, typically in the late thirties or early forties, in that men are affected as frequently as women, and in that the initial disease activity is often in the spinal cord and not in the brain. PPMS often migrates into the brain, but is less likely to damage brain areas than RRMS or SPMS. For example, people with PPMS are less likely to develop cognitive problems than those with RRMS or SPMS. PPMS is the subtype of MS that is least likely to show inflammatory (gadolinium enhancing) lesions on MRI scans however, recent trials have demonstrated that these do occur (Hawker Ann Neurol 2009). The Primary Progressive form of the disease affects between 10 and 15% of all people with multiple sclerosis. PPMS may be defined according to the criteria in McDonald et al. Ann Neurol 50:121-7 (2001). (Polman et al 2010 Diagnostic Criteria for Multiple Sclerosis:2010 Revisions to the McDonald Criteria ANN NEUROL 2011; 69:292-302) The subject with PPMS treated herein is usually one with probable or definitive diagnosis of PPMS.

“Secondary progressive multiple sclerosis” or “SPMS” is characterized by following an initial RRMS disease course with progression, with or without occasional relapses, minor remissions, and periods of stagnation or plateaus. SPMS may be seen as a long-term outcome of RRMS in that most SPMS patients initially begin with RR disease as defined herein. However, once the baseline between relapses begins to progressively detiororate, the patient has switched from RRMS to SPMS (Lublin, F. D. & Reingold, S. O (1996)). People who develop SPMS may have had a period of RRMS that lasted anything from two to forty years or more. From the onset of the secondary progressive phase of the disease, disability starts advancing much quicker than it did during RRMS though the progress can still be quite slow in some individuals. After 10 years, 50% of people with RRMS will have developed SPMS. SPMS tends to be associated with lower levels of inflammatory lesion formation than in RRMS but the total burden of disease continues to progress. At any one time, SPMS accounts around 30% of all people with multiple sclerosis.

“Progressive relapsing multiple sclerosis” refers to “PRMS” is characterized by progressive disease from onset, with clear acute relapses, with or without full recovery; periods between relapses characterized by continuing progression. PRMS is an additional, albeit rare, clinical course (Lublin, F. D. & Reingold, S. O (1996). PRMS affects around 5% of all people with multiple sclerosis. Some neurologists believe PRMS is a variant of PPMS and patients with PRMS are often considered to have the same prognosis as those with PPMS.

Multiple sclerosis may also be defined as “benign MS” or “malignant MS”. Benign MS may be defined by a disease state in which the patient remains fully functional in all neurologic systems. Typically, this may last for about 15 years after disease onset. Malignant MS may be defined as a disease state characterized by a rapid progressive course, leading to significant disability in multiple neurologic systems or death in a relatively short time after disease onset course (Lublin, F. D. & Reingold, S. O (1996)).

While MS has long been considered a T-cell-mediated disease, there is increasing emphasis on and understanding of the role of B cells in MS. In both open-label and controlled clinical studies in RRMS patients, depletion of B cells with anti-CD20 MAbs (i.e., rituximab and ocrelizumab) resulted in significantly decreased inflammatory lesions and relapse rates (Hauser et al, 2008; Bar-Or et al, 2008; Kappos et al, 2011). The efficacy demonstrated in these clinical studies with B-cell depleting MAbs has confirmed the importance of B cells in MS pathogenesis. Rituximab and ocrelizumab deplete B cells that express CD20, while antibodies for use according to the present invention target B cells that express CD 19. However, there remains an unmet clinical need for alternative treatments. The present invention provides for the use of an anti-CD19 antibody in the treatment of multiple sclerosis. Conceptually it is to be expected that an anti-CD19 antibody capable of depleting B cells would have all the advantages of the anti-CD20 antibodies known in the art for the treatment of MS, given that CD19 is expressed on all B cells that express CD20, however, targeting CD19 is likely to confer additional advantages because it is also expressed on B cells that do not express, or do not express substantial levels of, CD20. The broader range of B-cell subsets that express CD19 include earlier stage pre-cursor cells and later stage differentiated cells including plasmablasts and some plasma cells, which are the major source of antibody production (Dalakas, 2008; Tedder, 2009).

The expression of CD19 on this broader range of B cells is important in the context of treating MS because, mechanistically, B cells appear to have both antibody-dependent and antibody-independent roles in MS. This is based on the observation of B cells, antibody-secreting plasma cells, and auto-antibodies to CNS components in the cerebrospinal fluid (CSF) and central nervous system (CNS) of patients with MS with >90% of MS patients having oligoclonal bands of immunoglobulins in their CSF. Therefore, by targeting this broader range of B cells, a B cell depleting anti-CD19 antibody is likely to have greater impact in the treatment of MS as it kills also plasmablast and plasma cells, the more proximate antibody producing cells in MS. In addition, plasmablasts which are CD20−, CD19 and CD138+ have been suggested to be the main effector B-cell population involved in on-going active inflammation in patients with MS (Cepok S et al. (2005). Brain 128(Pt 7):1667-76).

Additionally, B cells may play a role in antigen presentation in the CNS and in priming of naive CNS reactive T cells as part of the MS disease process (Sellebjerg et al, 1998). Evidence for the antigen-presenting cell function of B cells in the CNS stems from studies showing that B cells and plasma cells in the CNS have undergone rapid and extensive T cell-mediated, antigen-driven clonal expansion and somatic hypermutation (Qin et al, 2003; Monson et al, 2005). Importantly, CD19+CD20− short-lived plasmablasts have been suggested as being the main effector B-cell population involved in on-going active inflammation in patients with MS (Cepok et al, 2005). Additionally, CNS resident B cells also contribute to the production of pro-inflammatory cytokines which attract and support survival of other destructive immune cells. Krzysiek and colleagues (Krzysiek et al, 1999) observed that B-cell receptor signalling induces expression of the two T-cell chemokines, macrophage inflammatory protein (MIP)-1a and MIP-1b, by naive, memory, and germinal centre B cells. Recent studies have suggested that the lymphoid neogenesis observed in the meningeal and sub-meningeal layers of the brain in MS is likely driven by cytokines and chemokines in the adjacent microenvironment. Corcione and colleagues (Corcione et al, 2004) identified lymphotoxin-a, CXCL12, and CXCL13 in the CSF and CNS tissue of MS patients. Additionally, B-cell activating factor (BAFF) of the tumour-necrosis factor family expression is significantly up-regulated in MS brain lesions (Krumbholz et al, 2005). Although the role of Epstein-Barr virus in MS remains controversial, there are reports of latently infected B cells detectable in white matter lesions. The expression of latency proteins LMP-1 and LMP-2A promotes the survival and differentiation of B cells and may contribute to dysregulation of these cells in MS resulting in amplification of the disease process by enhancing antibody production and antigen presentation to CD4 and CD8 T cells (Serafini et al, 2010).

Experimental autoimmune encephalomyelitis (EAE) is an animal model of MS induced by the immunization with myelin components or spinal cord homogenates from diseased animals resulting in the generation of a CNS-directed immune response. In EAE models, depletion of B cells during active disease with anti-CD20 MAb has been shown to dramatically suppress EAE symptoms by 50% to 100% (Matsushita et al, 2008; Monson et al, 2011). These rodent data were supported by B-cell depletion preventing both clinical manifestations and pathological findings of the disease in a marmoset model of human myelin oligodendrocyte glycoprotein-induced EAE (Kap et al, 2010). Currently, the human experience with CD20 B-cell depletion from clinical trials provides the best evidence for the importance of B cells in MS and the utility of targeting these cells to improve outcomes (Hauser et al, 2008; Bar-Or et al, 2008; Kappos et al, 2011).

Based on the presence or absence of relapses and remissions or progression of neurologic deficits, MS patients may be categorized into one of four clinical types. Primary progressive (PP) MS presents with “disease progression from the onset with occasional plateaus and temporary minor improvements” but without relapses or remissions during its course. Secondary progressive (SP) MS patients begin with a pattern of relapsing-remitting (RR) MS that later undergoes a transition to a progressive course with or without superimposed relapses. PP patients are reported to differ from those with RR and SP MS in their clinical, genetic, laboratory, imaging, and pathologic characteristics, as well as in their response to therapeutic agents. The incidence of PP type is reported to be between 8 and 37% among patients with MS. PPMS is relatively more common in patients who present at a later age (after the age of 40 years) and is more common in men. The most common presentation in PP disease is a chronic progressive myelopathy. Pathologically, PPMS has less perivascular cuffing and parenchymal cellular infiltration compared with SPMS. PPMS has a prognosis of significant and severe disability, and no therapeutic intervention has been proved to arrest or slow its relentlessly progressive course.

Recent pathological studies offer key insights into the potential role of plasma cells and plasmablasts and the potential role of such CD19 positive but CD20 negative B-cell lineage derived cells.

In the Frischer study (Brain 2009) both T- and B-cell infiltrates correlated with the activity of demyelinating lesions, while clearly plasma cell (CD19+ and CD20−) infiltrates were most pronounced in patients with secondary progressive multiple sclerosis (SPMS) and primary progressive multiple sclerosis. These plasma cell infiltrates persisted, when T- and B-cell infiltrates declined over time to levels observed in age matched controls. A significant association between inflammation and axonal injury was seen in the overall multiple sclerosis population as well as in progressive forms of multiple sclerosis.

In short, advances in the understanding of B cells and their role in the pathophysiology of MS provide a strong rationale for B-cell-targeted therapies and anti-CD 19 may have an additional effect on the establishment of secondary progressive MS and may have unique ability to target the CD19+CD20− B-cells involved in PPMS.

The present disclosure provides a method of treating multiple sclerosis in a subject suffering there from, comprising administering to the subject an effective amount of human, humanized, or chimeric anti-CD19 antibodies that bind to a CD19 antigen. The human, humanized, or chimeric anti-CD19 antibody may mediate ADCC, CDC, and/or apoptosis in an amount sufficient to deplete circulating B cells.

### Terminology

Before continuing to describe the present disclosure in further detail, it is to be understood that this disclosure is not limited to specific compositions or process steps, as such may vary. It must be noted that, as used in this specification and the appended claims, the singular form “a”, “an” and “the” include plural referents unless the context clearly dictates otherwise.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the terms “antibody” and “antibodies” (immunoglobulins) encompass monoclonal antibodies (including full-length monoclonal antibodies), polyclonal antibodies, multispecific antibodies (e.g., bispecific antibodies) formed from at least two intact antibodies, human antibodies, humanized antibodies, camelid antibodies, chimeric antibodies, single-chain Fvs (scFv), single-chain antibodies, single domain antibodies, domain antibodies, Fab fragments, F(ab′)2 fragments, antibody fragments that exhibit the desired biological activity, disulphide-linked Fvs (sdFv), and anti-idiotypic (anti-Id) antibodies (including, e.g., anti-Id antibodies to antibodies of the disclosure), intrabodies, and epitope-binding fragments of any of the above. In particular, antibodies include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

Native antibodies are usually heterotetrameric glycoproteins of about 150,000 Daltons, composed of two identical light (L) chains and two identical heavy (H) chains. Each light chain is linked to a heavy chain by one covalent disulphide bond, while the number of disulphide linkages varies between the heavy chains of different immunoglobulin isotypes. Each heavy and light chain also has regularly spaced intrachain disulphide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Light chains are classified as either lambda chains or kappa chains based on the amino acid sequence of the light chain constant region. The variable domain of a kappa light chain may also be denoted herein as VK. The term “variable region” may also be used to describe the variable domain of a heavy chain or light chain. Particular amino acid residues are believed to form an interface between the light and heavy chain variable domains. Such antibodies may be derived from any mammal, including, but not limited to, humans, monkeys, pigs, horses, rabbits, dogs, cats, mice, etc.

The term “variable” refers to the fact that certain portions of the variable domains differ extensively in sequence among antibodies and are responsible for the binding specificity of each particular antibody for its particular antigen. However, the variability is not evenly distributed through the variable domains of antibodies. It is concentrated in segments called Complementarity Determining Regions (CDRs) both in the light chain and the heavy chain variable domains. The more highly conserved portions of the variable domains are called the framework regions (FW). The variable domains of native heavy and light chains each comprise four FW regions, largely adopting a β-sheet configuration, connected by three CDRs, which form loops connecting, and in some cases forming part of, the β-sheet structure. The CDRs in each chain are held together in close proximity by the FW regions and, with the CDRs from the other chain, contribute to the formation of the antigen-binding site of antibodies (see, Kabat et al., Sequences of Proteins of Immunological Interest, 5th Ed. Public Health Service, National Institutes of Health, Bethesda, Md. (1991)). The constant domains are generally not involved directly in antigen binding, but may influence antigen binding affinity and may exhibit various effector functions, such as participation of the antibody in ADCC, CDC, and/or apoptosis.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, monoclonal antibodies are advantageous in that they can be synthesized by hybridoma cells that are uncontaminated by other immunoglobulin producing cells. Alternative production methods are known to those trained in the art, for example, a monoclonal antibody may be produced by cells stably or transiently transfected with the heavy and light chain genes encoding the monoclonal antibody.

The term “chimeric” antibodies includes antibodies in which at least one portion of the heavy and/or light chain is identical with or homologous to corresponding sequences in antibodies derived from a particular species or belonging to a particular antibody class or subclass, and at least one other portion of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable domain antigen-binding sequences derived from a nonhuman primate (e.g., Old World Monkey, such as baboon, rhesus or cynomolgus monkey) and human constant region sequences (U.S. Pat. No. 5,693,780).

“Humanized” forms of nonhuman (e.g., murine) antibodies are chimeric antibodies that contain minimal sequence derived from nonhuman immunoglobulin. For the most part, humanized antibodies are human immunoglobulins (recipient antibody) in which the native CDR residues are replaced by residues from the corresponding CDR of a nonhuman species (donor antibody) such as mouse, rat, rabbit or nonhuman primate having the desired specificity, affinity, and capacity. In some instances, FW region residues of the human immunoglobulin are replaced by corresponding nonhuman residues. Furthermore, humanized antibodies may comprise residues that are not found in the recipient antibody or in the donor antibody. These modifications are made to further refine antibody performance. In general, a humanized antibody heavy or light chain will comprise substantially all of at least one or more variable domains, in which all or substantially all of the CDRs correspond to those of a nonhuman immunoglobulin and all or substantially all of the FWs are those of a human immunoglobulin sequence. In certain embodiments, the humanized antibody will comprise at least a portion of an immunoglobulin constant region (Fc), typically that of a human immunoglobulin. For further details, see, Jones et al., Nature, 321:522-525 (1986); Riechmann et al., Nature, 332:323-329 (1988); and Presta, Curr. Op. Struct. Biol., 2:593-596 (1992).

A “human antibody” can be an antibody derived from a human or an antibody obtained from a transgenic organism that has been “engineered” to produce specific human antibodies in response to antigenic challenge and can be produced by any method known in the art. In certain techniques, elements of the human heavy and light chain loci are introduced into strains of the organism derived from embryonic stem cell lines that contain targeted disruptions of the endogenous heavy chain and light chain loci. The transgenic organism can synthesize human antibodies specific for human antigens, and the organism can be used to produce human antibody-secreting hybridomas. A human antibody can also be an antibody wherein the heavy and light chains are encoded by a nucleotide sequence derived from one or more sources of human DNA. A fully human antibody also can be constructed by genetic or chromosomal transfection methods, as well as phage display technology, or in vitro activated B cells, all of which are known in the art.

The terms “Fc receptor” or “FcR” are used to describe a receptor that binds to the Fc region of an antibody. In one embodiment, the FcR is a native sequence human FcR. Moreover, in certain embodiments, the FcR is one which binds an IgG antibody (a gamma receptor) and includes receptors of the FcγRI, FcγRII, FcγRIII, and FcγRIV subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (See, Daeron, Annu Rev. Immunol., 15:203-234 (1997)). FcRs are reviewed in Ravetch and Kinet, Annu Rev. Immunol., 9:457-92 (1991); Capel et al., Immunomethods, 4:25-34 (1994); and de Haas et al., J. Lab. Clin. Med., 126:330-41 (1995). Other FcRs, including those to be identified in the future, are encompassed by the term “FcR” herein. The term also includes the neonatal receptor, FcRn, which is responsible for the transfer of maternal IgGs to the foetus (Guyer et al., Immunol., 117:587 (1976) and Kim et al., J. Immunol., 24:249 (1994)).

“Antibody-dependent cell-mediated cytotoxicity” and “ADCC” refer to a cell-mediated reaction in which non-specific cytotoxic cells (e.g., Natural Killer (NK) cells, neutrophils, and macrophages) recognize bound antibody on a target cell and subsequently cause lysis of the target cell. In one embodiment, such cells are human cells. While not wishing to be limited to any particular mechanism of action, these cytotoxic cells that mediate ADCC generally express Fc receptors (FcRs). The primary cells for mediating ADCC, NK cells, express FcγRIII, whereas monocytes express FcγRI, FcγRII, FcγRIII and/or FcγRIV. FcR expression on hematopoietic cells is summarized in Ravetch and Kinet, Annu Rev. Immunol., 9:457-92 (1991). To assess ADCC activity of a molecule, an in vitro ADCC assay, such as that described in U.S. Pat. Nos. 5,500,362 or 5,821,337 may be performed. Useful effector cells for such assays include peripheral blood mononuclear cells (PBMC) and Natural Killer (NK) cells. Alternatively, or additionally, ADCC activity of the molecules of interest may be assessed in vivo, e.g., in an animal model such as that disclosed in Clynes et al., Proc. Natl. Acad. Sci. (USA), 95:652-656 (1998).

“Effector cells” are leukocytes which express one or more FcRs and perform effector functions. The cells express at least FcγRI, FCγRII, FcγRIII and/or FcγRIV and carry out ADCC effector function. Examples of human leukocytes which mediate ADCC include peripheral blood mononuclear cells (PBMC), natural killer (NK) cells, monocytes, cytotoxic T cells and neutrophils.

“Complement dependent cytotoxicity” and “CDC” refer to the lysing of a target cell in the presence of complement. The complement activation pathway is initiated by the binding of the first component of the complement system (C1q) to a molecule, an antibody for example, complexed with a cognate antigen. To assess complement activation, a CDC assay, e.g. as described in Gazzano-Santoro et al., 1996, J. Immunol. Methods, 202:163, may be performed.

### Anti-CD19 Antibodies

The present disclosure relates to human, humanized, or chimeric anti-CD 19 antibodies that bind to the human CD19 antigen, as well as to compositions comprising such antibodies. In certain embodiments, a human, humanized, or chimeric anti-CD 19 antibody may mediate antigen-dependent-cell-mediated-cytotoxicity (ADCC). In other embodiments, the present disclosure is directed toward compositions comprising a human, humanized, or chimeric anti-CD19 antibody of the IgG1 and/or IgG3 human isotype, as well as to a human, humanized, or chimeric anti-CD19 antibody of the IgG2 and/or IgG4 human isotype, that may mediate human ADCC, CDC, and/or apoptosis. In further embodiments, a human, humanized, or chimeric anti-CD19 antibody may inhibit anti-IgM/CpG stimulated B cell proliferation.

By way of example, exemplary humanized antibodies that specifically bind to CD19 are provided herein. In certain exemplary embodiments, the anti-CD19 antibody comprises a heavy chain variable region, VH, comprising at least one CDR sequence selected from the group consisting of SEQ ID NO: 1, SEQ ID NO: 2, and SEQ ID NO: 3. In certain embodiments, the VH comprises a CDR1 sequence comprising an amino sequence of SEQ ID NO: 1, a CDR2 sequence comprising an amino sequence of SEQ ID NO: 2, and CDR3 sequence comprising an amino sequence of SEQ ID NO: 3. In additional embodiments, the anti-CD19 antibody comprises a heavy chain variable region, VL, comprising at least one CDR sequence selected from the group consisting of SEQ ID NO: 4, SEQ ID NO: 5 and SEQ ID NO: 6. In some embodiments, the VL comprises a CDR1 sequence comprising an amino sequence of SEQ ID NO: 4, a CDR2 sequence comprising an amino sequence of SEQ ID NO: 5, and a CDR3 sequence comprising an amino sequence of SEQ ID NO: 6.

In some embodiments, the anti-CD19 antibody comprises a VH comprising an amino acid sequence of SEQ ID NO: 7, and a VL comprising an amino acid sequence of SEQ ID NO: 8.

In certain exemplary embodiments, the anti-CD19 antibody comprises a heavy chain variable region, VH, comprising at least one CDR sequence selected from the group consisting of SEQ ID NO: 9, SEQ ID NO: 10 and SEQ ID NO: 11. In certain embodiments, the VH comprises a CDR1 sequence comprising an amino sequence of SEQ ID NO: 9, a CDR2 sequence comprising an amino sequence of SEQ ID NO: 10 and CDR3 sequence comprising an amino sequence of SEQ ID NO: 11. In additional embodiments, the anti-CD19 antibody comprises a heavy chain variable region, VL, comprising at least one CDR sequence selected from the group consisting of SEQ ID NO: 12, SEQ ID NO: 13 and SEQ ID NO: 14. In some embodiments, the VL comprises a CDR1 sequence comprising an amino sequence of SEQ ID NO: 12, a CDR2 sequence comprising an amino sequence of SEQ ID NO: 13 and a CDR3 sequence comprising an amino sequence of SEQ ID NO: 14.

In some embodiments, the anti-CD19 antibody comprises a VH comprising an amino acid sequence of SEQ ID NO: 15, and a VL comprising an amino acid sequence of SEQ ID NO: 16.

### Variant Fc Regions

It is known that variants of the Fc region (e.g., amino acid substitutions and/or additions and/or deletions) enhance or diminish effector function of the antibody (See e.g., U.S. Pat. Nos. 5,624,821; 5,885,573; 6,538,124; 7,317,091; 5,648,260; 6,538,124; WO 03/074679; WO 04/029207; WO 04/099249; WO 99/58572; US Publication No. 2006/0134105; 2004/0132101; 2006/0008883) and may alter the pharmacokinetic properties (e.g. half-life) of the antibody (see, U.S. Pat. Nos. 6,277,375 and 7,083,784). Thus, in certain embodiments, the anti-CD19 antibodies of the present disclosure comprise an altered Fc region (also referred to herein as “variant Fc region”) in which one or more alterations have been made in the Fc region in order to change functional and/or pharmacokinetic properties of the antibodies. Such alterations may result in a decrease or increase of C1q binding and complement dependent cytotoxicity (CDC) or of FcγR binding, for IgG, and antibody-dependent cellular cytotoxicity (ADCC), or antibody dependent cell-mediated phagocytosis (ADCP). The present disclosure encompasses the antibodies described herein with variant Fc regions wherein changes have been made to fine tune the effector function, and providing a desired effector function. Accordingly, in certain embodiments of the present disclosure, the anti-CD19 antibodies of the present disclosure comprise a variant Fc region (i.e., Fc regions that have been altered as discussed below). Anti-CD19 antibodies of the present disclosure comprising a variant Fc region are also referred to here as “Fc variant antibodies.” As used herein native refers to the unmodified parental sequence and the antibody comprising a native Fc region is herein referred to as a “native Fc antibody”. Fc variant antibodies can be generated by numerous methods well known to one skilled in the art. Non-limiting examples include, isolating antibody coding regions (e.g., from hybridoma) and making one or more desired substitutions in the Fc region of the isolated antibody coding region. Alternatively, the antigen-binding portion (e.g., variable regions) of an anti-CD19 antibody may be sub-cloned into a vector encoding a variant Fc region. In certain embodiments, the variant Fc region exhibits a similar level of inducing effector function as compared to the native Fc region. In another embodiment, the variant Fc region exhibits a higher induction of effector function as compared to the native Fc. Some specific embodiments of variant Fc regions are detailed herein. Methods for measuring effector function are well known in the art.

It is understood that the Fc region as used herein includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as set forth in Kabat. Fc may refer to this region in isolation, or this region in the context of an antibody, antibody fragment, or Fc fusion protein. Polymorphisms have been observed at a number of different Fc positions, including but not limited to positions 270, 272, 312, 315, 356, and 358 as numbered by the EU index, and thus slight differences between the presented sequence and sequences in the prior art may exist.

The present disclosure encompasses Fc variant proteins which have altered binding properties for an Fc ligand (e.g., an Fc receptor, C1q) relative to a comparable molecule (e.g., a molecule having a wild-type Fc sequence, or a molecule having a non-variant Fc sequence). Examples of binding properties include but are not limited to, binding specificity, equilibrium dissociation constant (K_{D}), dissociation and association rates (k_{off} and kₒₙ respectively), binding affinity and/or avidity. It is generally understood that a binding molecule (e.g., an Fc variant protein such as an antibody) with a low K_{D} may be preferable to a binding molecule with a high K_{D}. However, in some instances the value of the kₒₙ, or k_{off} may be more relevant than the value of the K_{D}. One skilled in the art can determine which kinetic parameter is most important for a given antibody application.

The affinities and binding properties of an Fc domain for its ligand may be determined by a variety of in vitro assay methods (biochemical or immunological based assays) known in the art for determining Fc-FcγR interactions, i.e., specific binding of an Fc region to an FcγR including but not limited to, equilibrium methods (e.g., enzyme-linked immunoabsorbent assay (ELISA), or radioimmunoassay (RIA)), or kinetics (e.g., BIACORE™ analysis), and other methods such as indirect binding assays, competitive inhibition assays, fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions.

In some embodiments, the Fc variant protein has enhanced binding to one or more Fc ligand relative to a comparable molecule. In another embodiment, the Fc variant protein has an affinity for an Fc ligand that is at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold greater than that of a comparable molecule. In a specific embodiment, the Fc variant protein has enhanced binding to an Fc receptor. In another specific embodiment, the Fc variant protein has enhanced binding to the Fc receptor FcγRIIIA. In a further specific embodiment, the Fc variant protein has enhanced biding to the Fc receptor FcγRIIB. In still another specific embodiment, the Fc variant protein has enhanced binding to the Fc receptor FcRn. In yet another specific embodiment, the Fc variant protein has enhanced binding to C1q relative to a comparable molecule.

In some embodiments, an anti-CD19 antibody of the disclosure comprises a variant Fc domain wherein said variant Fc domain has enhanced binding affinity to Fc gamma receptor IIB relative to a comparable non-variant Fc domain. In a further embodiment, an anti-CD19 antibody of the disclosure comprises a variant Fc domain wherein said variant Fc domain has an affinity for Fc gamma receptor IIB that is at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold greater than that of a comparable non-variant Fc domain.

In some embodiments, the Fc variant protein has reduced binding to one or more Fc ligand relative to a comparable molecule. In another embodiment, the Fc variant protein has an affinity for an Fc ligand that is at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold lower than that of a comparable molecule. In a specific embodiment, the Fc variant protein has reduced binding to an Fc receptor. In another specific embodiment, the Fc variant protein has reduced binding to the Fc receptor FcγRIIIA. In a further specific embodiment, an Fc variant described herein has an affinity for the Fc receptor FcγRIIIA that is at least about 5 fold lower than that of a comparable molecule, wherein said Fc variant has an affinity for the Fc receptor FcγRIIB that is within about 2 fold of that of a comparable molecule. In still another specific embodiment, the Fc variant protein has reduced binding to the Fc receptor FcRn. In yet another specific embodiment, the Fc variant protein has reduced binding to C1q relative to a comparable molecule.

The ability of any particular Fc variant protein to mediate lysis of the target cell by ADCC can be assayed. To assess ADCC activity an Fc variant protein of interest is added to target cells in combination with immune effector cells, which may be activated by the antigen antibody complexes resulting in cytolysis of the target cell. Cytolysis is generally detected by the release of label (e.g. radioactive substrates, fluorescent dyes or natural intracellular proteins) from the lysed cells. Useful effector cells for such assays include peripheral blood mononuclear cells (PBMC) and Natural Killer (NK) cells. Specific examples of in vitro ADCC assays are described in Wisecarver et al., 1985 79:277-282; Bruggemann et al., 1987, J Exp Med 166:1351-1361; Wilkinson et al., 2001, J Immunol Methods 258:183-191; Patel et al., 1995 J Immunol Methods 184:29-38. ADCC activity of the Fc variant protein of interest may also be assessed in vivo, e.g., in an animal model such as that disclosed in Clynes et al., 1998, Proc. Natl. Acad. Sci. USA 95:652-656.

In some embodiments, an Fc variant protein has enhanced ADCC activity relative to a comparable molecule. In a specific embodiment, an Fc variant protein has ADCC activity that is at least 2 fold, or at least 3 fold, or at least 5 fold or at least 10 fold or at least 50 fold or at least 100 fold greater than that of a comparable molecule. In another specific embodiment, an Fc variant protein has enhanced binding to the Fc receptor FcγRIIIA and has enhanced ADCC activity relative to a comparable molecule. In other embodiments, the Fc variant protein has both enhanced ADCC activity and enhanced serum half-life relative to a comparable molecule.

In some embodiments, an Fc variant protein has reduced ADCC activity relative to a comparable molecule. In a specific embodiment, an Fc variant protein has ADCC activity that is at least 2 fold, or at least 3 fold, or at least 5 fold or at least 10 fold or at least 50 fold or at least 100 fold lower than that of a comparable molecule. In another specific embodiment, an Fc variant protein has reduced binding to the Fc receptor FcγRIIIA and has reduced ADCC activity relative to a comparable molecule. In other embodiments, the Fc variant protein has both reduced ADCC activity and enhanced serum half-life relative to a comparable molecule.

In some embodiments, the present disclosure provides Fc variants, wherein the Fc region comprises a non-naturally occurring amino acid residue at one or more positions selected from the group consisting of 234, 235, 236, 237, 238, 239, 240, 241, 243, 244, 245, 247, 251, 252, 254, 255, 256, 262, 263, 264, 265, 266, 267, 268, 269, 279, 280, 284, 292, 296, 297, 298, 299, 305, 313, 316, 325, 326, 327, 328, 329, 330, 331, 332, 333, 334, 339, 341, 343, 370, 373, 378, 392, 416, 419, 421, 440 and 443 as numbered by the EU index as set forth in Kabat. Optionally, the Fc region may comprise a non-naturally occurring amino acid residue at additional and/or alternative positions known to one skilled in the art (see, e.g., U.S. Pat. Nos. 5,624,821; 6,277,375; 6,737,056; PCT Patent Publications WO 01/58957; WO 02/06919; WO 04/016750; WO 04/029207; WO 04/035752; WO 04/074455; WO 04/099249; WO 04/063351; WO 05/070963; WO 05/040217, WO 05/092925 and WO 06/020114).

In certain embodiments, the present disclosure provides an Fc variant, wherein the Fc region comprises at least one non naturally occurring amino acid residue selected from the group consisting of 234D, 234E, 234N, 234Q, 234T, 234H, 234Y, 234I, 234V, 234F, 235A, 235D, 235R, 235W, 235P, 235S, 235N, 235Q, 235T, 235H, 235Y, 235I, 235V, 235F, 236E, 239D, 239E, 239N, 239Q, 239F, 239T, 239H, 239Y, 240I, 240A, 240T, 240M, 241W, 241L, 241Y, 241E, 241R, 243W, 243L, 243Y, 243R, 243Q, 244H, 245A, 247L, 247V, 247G, 251F, 252Y, 254T, 255L, 256E, 256M, 262I, 262A, 262T, 262E, 263I, 263A, 263T, 263M, 264L, 264I, 264W, 264T, 264R, 264F, 264M, 264Y, 264E, 265G, 265N, 265Q, 265Y, 265F, 265V, 265I, 265L, 265H, 265T, 266I, 266A, 266T, 266M, 267Q, 267L, 268E, 269H, 269Y, 269F, 269R, 270E, 280A, 284M, 292P, 292L, 296E, 296Q, 296D, 296N, 296S, 296T, 296L, 296I, 296H, 269G, 297S, 297D, 297E, 298H, 298I, 298T, 298F, 299I, 299L, 299A, 299S, 299V, 299H, 299F, 299E, 305I, 313F, 316D, 325Q, 325L, 325I, 325D, 325E, 325A, 325T, 325V, 325H, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 330I, 330F, 330R, 330H, 331G, 331A, 331L, 331M, 331F, 331W, 331K, 331Q, 331E, 331S, 331V, 331I, 331C, 331Y, 331H, 331R, 331N, 331D, 331T, 332D, 332S, 332W, 332F, 332E, 332N, 332Q, 332T, 332H, 332Y, 332A, 339T, 370E, 370N, 378D, 392T, 396L, 416G, 419H, 421K, 440Y and 434W as numbered by the EU index as set forth in Kabat. Optionally, the Fc region may comprise additional and/or alternative non-naturally occurring amino acid residues known to one skilled in the art (see, e.g., U.S. Pat. Nos. 5,624,821; 6,277,375; 6,737,056; PCT Patent Publications WO 01/58957; WO 02/06919; WO 04/016750; WO 04/029207; WO 04/035752 and WO 05/040217).

### Glycosylation of Antibodies

Many polypeptides, including antibodies, are subjected to a variety of post-translational modifications involving carbohydrate moieties, such as glycosylation with oligosaccharides. There are several factors that can influence glycosylation. The species, tissue and cell type have all been shown to be important in the way that glycosylation occurs. In addition, the extracellular environment, through altered culture conditions such as serum concentration, may have a direct effect on glycosylation. (Lifely et al., 1995, Glycobiology 5(8): 813-822).

All antibodies contain carbohydrate at conserved positions in the constant regions of the heavy chain. Each antibody isotype has a distinct variety of N-linked carbohydrate structures. IgG has a single N-linked biantennary carbohydrate at Asn297 of the CH2 domain. For IgG from either serum or produced ex vivo in hybridomas or engineered cells, the IgG are heterogeneous with respect to the Asn297 linked carbohydrate (Jefferis et al., 1998, Immunol. Rev. 163:59-76; Wright et al., 1997, Trends Biotech 15:26-32, both incorporated entirely by reference). For human IgG, the core oligosaccharide normally consists of GlcNAc₂Man₃GlcNAc, with differing numbers of outer residues.

The carbohydrate moieties of the present disclosure will be described with reference to commonly used nomenclature for the description of oligosaccharides. A review of carbohydrate chemistry which uses this nomenclature is found in Hubbard et al. 1981, Ann. Rev. Biochem. 50:555-583, incorporated entirely by reference. This nomenclature includes, for instance, Man, which represents mannose; GlcNAc, which represents 2-N-acetylglucosamine; Gal which represents galactose; Fuc for fucose; and Glc, which represents glucose. Sialic acids are described by the shorthand notation NeuNAc, for 5-N-acetylneuraminic acid, and NeuNGc for 5-glycolylneuraminic.

The present disclosure contemplates antibodies that comprise modified glycoforms or engineered glycoforms. By “modified glycoform” or “engineered glycoform” as used herein is meant a carbohydrate composition that is covalently attached to a protein, for example an antibody, wherein said carbohydrate composition differs chemically from that of a parent protein. Engineered glycoforms may be useful for a variety of purposes, including but not limited to enhancing or reducing FcγR-mediated effector function. In some embodiment, the antibodies of the present disclosure are modified to reduce the level of fucosylated oligosaccharides that are covalently attached to the Fc region. Antibodies having reduced level of fucosylated oligosaccharides covalently attached to the Fc region have been demonstrated to have increased ADCC activity.

A variety of methods are well known in the art for generating modified glycoforms (Shinkawa et al., 2003, J Biol Chem 278:3466-3473); (PCT WO 01/29246A1; PCT WO 02/31140A1; PCT WO 02/30954A1); Yamane-Ohnuki et al., 2004, Biotechnology and Bioengineering 87(5):614-621; (Potelligent™ technology [Biowa, Inc., Princeton, N.J.]; all of which are expressly incorporated by reference). These techniques control the level of fucosylated oligosaccharides that are covalently attached to the Fc region, for example by expressing an IgG in various organisms or cell lines, engineered or otherwise (for example Lec-13 CHO cells or rat hybridoma YB2/0 cells), or by regulating enzymes involved in the glycosylation pathway (for example FUT8 [α1,6-fucosyltranserase].

The present disclosure provides a composition comprising a plurality of glycosylated monoclonal anti-CD19 antibodies having an Fc region, wherein about 51-100% of the glycosylated anti-CD19 antibodies comprise an afucosylated core carbohydrate structure at Asn297 of the CH2 domain, e.g., a core carbohydrate structure that lacks fucose. In some embodiments, about 80-100%, or about 90-99%, or about 100% of the glycosylated antibodies comprise an afucosylated core carbohydrate structure at Asn297 of the CH2 domain.

The foregoing are examples of anti-CD19 antibodies for use in the methods of the present disclosure. Additional exemplary antibodies are provided in United States publication 2008-0138336, the disclosure of which is hereby incorporated by reference in its entirety.

The anti-CD19 antibodies described herein may efficiently deplete B cells expressing a recombinant human CD19 molecule in an hCD19 transgenic mouse model system (see e.g., Yazawa et al, Proc Natl Acad Sci USA. 102(42):15178-83 (2005) and Herbst et al., 335(1):213-22 (2010)). In certain embodiments, an anti-CD19 antibody of the disclosure may deplete circulating B cells, blood B cells, splenic B cells, marginal zone B cells, follicular B cells, peritoneal B cells, and/or bone marrow B cells. In certain embodiments, an anti-CD19 antibody of the present disclosure may achieve depletion of progenitor B cells, early pro-B cells, late pro-B cells, large-pre-B cells, small pre-B cells, immature B cells, mature B cells, antigen stimulated B cells, and/or plasma cells. In certain embodiments, B cell depletion by an anti-CD19 antibody of the present disclosure may persist for at least 1 day, at least 2 days, at least 3 days, at least 4 days, at least 5 days at least 6 days, at least 7 days, at least 8 days, at least 9 days, at least 10 days, at least 15 days, at least 20 days, at least 25 days, or at least 30 days. In some embodiments, B cell depletion by an anti-CD19 antibody of the present disclosure may persist for at least 1 week, at least 2 weeks, at least 3 weeks, at least 4 weeks, at least 5 weeks, at least 6 weeks, at least 7 weeks, at least 8 weeks, at least 9 weeks, or at least 10 weeks. In some embodiments, B cell depletion by an anti-CD19 antibody of the present disclosure may persist for at least 1 month, at least 2 months, at least 3 months, at least 4 months, at least 5 months, at least 6 months, at least 7 months, at least 8 months, at least 9 months, at least 10 months, at least 11 months or at least 12 months.

The anti-CD19 antibodies described herein may also efficiently deplete B cells in a human subject. In certain embodiments, an anti-CD19 antibody of the present disclosure may achieve at least about 20%, at least about 30%, at least about 40%, at least about 50%, at least about 60%, at least about 70%, at least about 80%, at least about 90%, at least about 95%, or about 100% B cell depletion. In some embodiments, an anti-CD19 antibody of the present disclosure may deplete B cell subsets in a human subject. In some embodiments, an anti-CD19 antibody of the present disclosure may deplete circulating B cells, blood B cells, splenic B cells, marginal zone B cells, follicular B cells, peritoneal B cells, and/or bone marrow B cells. CD 19 is present on the surface of B cells at all developmental stages. An anti-CD19 antibody may therefore deplete B cells of all developmental stages. In some embodiments, an anti-CD19 antibody of the present disclosure may achieve depletion of progenitor B cells, early pro-B cells, late pro-B cells, large-pre-B cells, small pre-B cells, immature B cells, mature B cells, antigen stimulated B cells, and/or plasma cells. Depletion of B cells may persist for extended periods of time. In certain embodiments, B cell depletion by an anti-CD 19 antibody of the present disclosure may persist for at least 1 day, at least 2 days, at least 3 days, at least 4 days, at least 5 days at least 6 days, at least 7 days, at least 8 days, at least 9 days, at least 10 days, at least 15 days, at least 20 days, at least 25 days, or at least 30 days. In certain embodiments, B cell depletion by an anti-CD19 antibody of the present disclosure may persist for at least 1 week, at least 2 weeks, at least 3 weeks, at least 4 weeks, at least 5 weeks, at least 6 weeks, at least 7 weeks, at least 8 weeks, at least 9 weeks, or at least 10 weeks. In certain embodiments, B cell depletion by an anti-CD19 antibody of the present disclosure may persist for at least 1 month, at least 2 months, at least 3 months, at least 4 months, at least 5 months, at least 6 months, at least 7 months, at least 8 months, at least 9 months, at least 10 months, at least 11 months or at least 12 months.

In some embodiments, an anti-CD19 antibody described herein depletes at least about 20%, at least about 30%, at least about 40%, at least about 50%, at least about 60%, at least about 70%, at least about 80%, at least about 90%, at least about 95%, or about 100% of circulating B cells, blood B cells, splenic B cells, marginal zone B cells, follicular B cells, peritoneal B cells, marrow B cells, progenitor B cells, early pro-B cells, late pro-B cells, large pre-B cells, small pre-B cells, immature B cells, mature B cells, antigen stimulated B cells, and/or plasma cells.

Depletion of B cells may persist for extended periods of time. In certain embodiments, B cell depletion by an anti-CD 19 antibody of the present disclosure may persist for at least 1 day, at least 2 days, at least 3 days, at least 4 days, at least 5 days at least 6 days, at least 7 days, at least 8 days, at least 9 days, at least 10 days, at least 15 days, at least 20 days, at least 25 days, or at least 30 days. In another embodiment, B cell depletion by an anti-CD19 antibody of the present disclosure may persist for at least 1 week, at least 2 weeks, at least 3 weeks, at least 4 weeks, at least 5 weeks, at least 6 weeks, at least 7 weeks, at least 8 weeks, at least 9 weeks, or at least 10 weeks. In a further embodiment, B cell depletion by an anti-CD19 antibody of the present disclosure may persist for at least 1 month, at least 2 months, at least 3 months, at least 4 months, at least 5 months, at least 6 months, at least 7 months, at least 8 months, at least 9 months, at least 10 months, at least 11 months or at least 12 months.

In certain embodiments, the anti-CD19 antibodies of the present disclosure mediate antibody-dependent cellular cytotoxicity (ADCC), complement-dependent cell-mediated cytotoxicity (CDC), and/or apoptosis. In certain embodiments, the anti-CD19 antibodies of the present disclosure mediate antibody-dependent cellular cytotoxicity (ADCC) and/or apoptosis. In certain embodiments, an anti-CD19 antibody of the present disclosure has enhanced antibody-dependent cellular cytotoxicity (ADCC). In certain embodiments, the anti-CD19 antibodies of the present comprise a variant Fc region that mediates enhanced antibody-dependent cellular cytotoxicity (ADCC). In certain embodiments, an anti-CD19 antibody of the present disclosure comprises an Fc region having complex N-glycoside-linked sugar chains linked to Asn297 in which fucose is not bound to N-acetylglucosamine in the reducing end, wherein said Fc region mediates enhanced antibody-dependent cellular cytotoxicity (ADCC).

### Immunoconjugates and Fusion Proteins

According to certain aspects of the present disclosure, therapeutic agents or toxins can be conjugated to chimerized, human, or humanized anti-CD19 antibodies for use in compositions and methods of the present disclosure. In certain embodiments, these conjugates can be generated as fusion proteins. Examples of therapeutic agents and toxins include, but are not limited to, members of the enediyne family of molecules, such as calicheamicin and esperamicin. Chemical toxins can also be taken from the group consisting of duocarmycin (see, e.g., U.S. Pat. No. 5,703,080 and U.S. Pat. No. 4,923,990), methotrexate, doxorubicin, melphalan, chlorambucil, ARA-C, vindesine, mitomycin C, cis-platinum, etoposide, bleomycin and 5-fluorouracil. Examples of chemotherapeutic agents also include Adriamycin, Doxorubicin, 5-Fluorouracil, Cytosine arabinoside (Ara-C), Cyclophosphamide, Thiotepa, Taxotere (docetaxel), Busulfan, Cytoxin, Taxol, Methotrexate, Cisplatin, Melphalan, Vinblastine, Bleomycin, Etoposide, Ifosfamide, Mitomycin C, Mitoxantrone, Vincreistine, Vinorelbine, Carboplatin, Teniposide, Daunomycin, Caminomycin, Aminopterin, Dactinomycin, Mitomycins, Esperamicins (see, U.S. Pat. No. 4,675,187), Melphalan, and other related nitrogen mustards.

In certain embodiments, anti-CD19 antibodies are conjugated to a cytostatic, cytotoxic or immunosuppressive agent wherein the cytotoxic agent is selected from the group consisting of an enediyne, a lexitropsin, a duocarmycin, a taxane, a puromycin, a dolastatin, a maytansinoid, and a vinca alkaloid. In certain, more specific embodiments, the cytotoxic agent is paclitaxel, docetaxel, CC-1065, SN-38, topotecan, morpholino-doxorubicin, rhizoxin, cyanomorpholino-doxorubicin, dolastatin-10, echinomycin, combretastatin, calicheamicin, maytansine, DM-1, auristatin E, AEB, AEVB, AEFP, MMAE (see, U.S. patent application Ser. No. 10/983,340), or netropsin.

In certain embodiments, the cytotoxic agent of an anti-CD19 antibody-cytotoxic agent conjugate of the present disclosure is an anti-tubulin agent. In specific embodiments, the cytotoxic agent is selected from the group consisting of a vinca alkaloid, a podophyllotoxin, a taxane, a baccatin derivative, a cryptophysin, a maytansinoid, a combretastatin, and a dolastatin. In other embodiments, the cytotoxic agent is vincristine, vinblastine, vindesine, vinorelbine, VP-16, camptothecin, paclitaxel, docetaxel, epithilone A, epithilone B, nocodazole, coichicine, colcimid, estramustine, cemadotin, discodermolide, maytansine, DM-1, AEFP, auristatin E, AEB, AEVB, AEFP, MMAE or eleutherobin.

In certain embodiments, an anti-CD19 antibody is conjugated to the cytotoxic agent via a linker, wherein the linker is a peptide linker. In other embodiments, an anti-CD19 antibody is conjugated to the cytotoxic agent via a linker, wherein the linker is a val-cit linker, a phe-lys linker, a hydrazone linker, or a disulphide linker.

In certain embodiments, the anti-CD19 antibody of an anti-CD19 antibody-cytotoxic agent conjugate is conjugated to the cytotoxic agent via a linker, wherein the linker is hydrolysable at a pH of less than 5.5. In a specific embodiment the linker is hydrolyzable at a pH of less than 5.0.

In certain embodiments, the anti-CD19 antibody of an anti-CD19 antibody-cytotoxic agent conjugate is conjugated to the cytotoxic agent via a linker, wherein the linker is cleavable by a protease. In a specific embodiment, the protease is a lysosomal protease. In other embodiments, the protease is, inter alia, a membrane-associated protease, an intracellular protease, or an endosomal protease.

In certain embodiments, the cytotoxic agent of an anti-CD19 antibody-cytotoxic agent conjugate is a tyrosine kinase inhibitor. Exemplary tyrosine kinase inhibitor compounds include ABT-869 (Abbott), Sutent (Pfizer), KI-20227 (Kirin Brewery), CYC-10268 (Cytopia), YM-359445 (Astellas Pharma), PLX-647 (Phenomix Corp./Plexxikon), JNJ-27301937 (Johnson & Johnson), and GW-2580 (GlaxoSmithKline).

In other embodiments, the tyrosine kinase inhibitor is a Syk inhibitor. An exemplary Syk inhibitor includes but is not limited to Fostamatinib. In some embodiments, the tyrosine kinase inhibitor is a Lyn inhibitor. An exemplary Lyn inhibitor includes but is not limited to bafetinib. In some embodiments, the tyrosine kinase inhibitor is Bruton's tyrosine kinase (Btk) inhibitor. An exemplary Btk inhibitor includes but is not limited to PCI-32765 (Pharmacyclics).

Other toxins that can be used in immunoconjugates of the present disclosure include poisonous lectins, plant toxins such as ricin, abrin, modeccin, botulina, and diphtheria toxins. Of course, combinations of the various toxins could also be coupled to one antibody molecule thereby accommodating variable cytotoxicity. Illustrative of toxins which are suitably employed in combination therapies of the disclosure are ricin, abrin, ribonuclease, DNase I, Staphylococcal enterotoxin-A, pokeweed anti-viral protein, gelonin, diphtherin toxin, Pseudomonas exotoxin, and Pseudomonas endotoxin. See, for example, Pastan et al., Cell, 47:641 (1986), and Goldenberg et al., Cancer Journal for Clinicians, 44:43 (1994). Enzymatically active toxins and fragments thereof which can be used include diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. See, for example, WO 93/21232 published Oct. 28, 1993.

Suitable toxins and chemotherapeutic agents are described in Remington's Pharmaceutical Sciences, 19th Ed. (Mack Publishing Co. 1995), and in Goodman And Gilman's The Pharmacological Basis of Therapeutics, 7th Ed. (MacMillan Publishing Co. 1985). Other suitable toxins and/or chemotherapeutic agents are known to those of skill in the art.

### Combination Therapy

An anti-CD19 immunotherapy described herein may be co-administered in combination with other B-cell surface receptor antibodies, including, but not limited to, anti-CD20 MAb, anti-CD52 MAb, anti-CD22 antibody, and anti-CD20 antibodies, such as RITUXAN™ (C2B8; RITUXIMAB™; IDEC Pharmaceuticals).

An anti-CD19 immunotherapy described herein may be co-administered in combination with an antibody specific for an Fc receptor selected from the group consisting of FcγRI, FcγRIIA, FcγRIIB, and/or FcγRIII. In certain embodiments, an anti-CD19 immunotherapy described herein may be administered in combination with an antibody specific for FcγRIIB. Anti-FcγRIIB antibodies suitable for this purpose have been described in US Patent Application Publication No. 2004185045, PCT Publication Nos. WO05051999A, WO05018669 and WO04016750.

In certain embodiments, an anti-CD19 and an anti-CD20 and/or an anti-CD22 mAb and/or an anti-CD52 mAb can be co-administered, optionally in the same pharmaceutical composition, in any suitable ratio. To illustrate, the ratio of the anti-CD19 and anti-CD20 antibody can be a ratio of about 1000:1, 500:1, 250:1, 100:1, 90:1, 80:1, 70:1, 60:1, 50:1, 40:1, 30:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50, 1:60, 1:70, 1:80, 1:90, 1:100, 1:250, 1:500 or 1:1000 or more. Likewise, the ratio of the anti-CD19 and anti-CD22 antibody can be a ratio of about 1000:1, 500:1, 250:1, 100:1, 90:1, 80:1, 70:1, 60:1, 50:1, 40:1, 30:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50, 1:60, 1:70, 1:80, 1:90, 1:100, 1:250, 1:500 or 1:1000 or more. Similarly, the ratio of the anti-CD19 and anti-CD52 antibody can be a ratio of about 1000:1, 500:1, 250:1, 100:1, 90:1, 80:1, 70:1, 60:1, 50:1, 40:1, 30:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50, 1:60, 1:70, 1:80, 1:90, 1:100, 1:250, 1:500 or 1:1000 or more.

An anti-CD19 immunotherapy for the treatment of MS described herein may also be co-administered in combination with one or more additional drugs that may be active in treating multiple sclerosis. These include interferon-betas, such as Avonex™ and Rebif™. Additional drugs that may be active in treating multiple sclerosis include Copaxone™; corticosteroids such as prednisone or methylprednisolone; immunosuppressive agents such as cyclosporine (or other calcineurin inhibitors, such as Prograf™); azathioprine, Rapamune™ and Cellcept™; anti-metabolites such as methotrexate; and antineoplastic agents such as mitoxantrone.

### Pharmaceutical Formulations

An anti-CD19 antibody composition may be formulated with a pharmaceutically acceptable carrier. The term “pharmaceutically acceptable” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also routinely contain compatible solid or liquid fillers, diluents or encapsulating substances which are suitable for administration into a human. When used in medicine, the salts should be pharmaceutically acceptable, but non-pharmaceutically acceptable salts may conveniently be used to prepare pharmaceutically acceptable salts thereof and are not excluded from the scope of the disclosure. Such pharmacologically and pharmaceutically acceptable salts include, but are not limited to, those prepared from the following acids: hydrochloric, hydrobromic, sulfuric, nitric, phosphoric, maleic, acetic, salicylic, citric, boric, formic, malonic, succinic, and the like. Also, pharmaceutically acceptable salts can be prepared as alkaline metal or alkaline earth salts, such as sodium, potassium or calcium salts. The term “carrier” denotes an organic or inorganic ingredient, natural or synthetic, with which the active ingredient is combined to facilitate the application. The components of the pharmaceutical compositions also are capable of being co-mingled with the antibodies of the present disclosure, and with each other, in a manner such that there is no interaction which would substantially impair the desired pharmaceutical efficacy.

According to certain aspects of the disclosure, anti-CD19 antibody compositions can be prepared for storage by mixing the antibody or immunoconjugate having the desired degree of purity with optional physiologically acceptable carriers, excipients or stabilizers (Remington's Pharmaceutical Sciences, 16th edition, Osol, A. Ed. (1999)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN, PLURONICS™ or polyethylene glycol (PEG).

The anti-CD19 antibody may be administered by any suitable means, including parenteral, intracranial, topical, subcutaneous, intraperitoneal, intrapulmonary, intranasal, and/or intralesional administration. Parenteral infusions include intramuscular, intravenous, intraarterial, or intraperitoneal. In addition, the antibody may suitably be administered by pulse infusion, e.g., with declining doses of the antibody. Preferably, the antibody may be administered intravenously, intracranially, subcutaneously or intrathecally, most preferably intravenously or subcutaneously.

Some of the pharmaceutical formulations include, but are not limited to:

(a) a sterile, preservative-free liquid concentrate for intravenous (i.v.) administration of anti-CD19 antibody, supplied at a concentration of 10 mg/ml in either 100 mg (10 mL) or 500 mg (50 mL) single-use vials. The product can be formulated for i.v. administration using sodium chloride, sodium citrate dihydrate, polysorbate and sterile water for injection. For example, the product can be formulated in 9.0 mg/mL sodium chloride, 7.35 mg/mL sodium citrate dihydrate, 0.7 mg/mL polysorbate 80, and sterile water for injection. The pH is adjusted to 6.5.

(b) A sterile, lyophilized powder in single-use glass vials for subcutaneous (s.c.) injection. The product can be formulated with sucrose, L-histidine hydrochloride monohydrate, L-histidine and polysorbate 20. For example, each single-use vial can contain 150 mg anti-CD19 antibody, 123.2 mg sucrose, 6.8 mg L-histidine hydrochloride monohydrate, 4.3 mg L-histidine, and 3 mg polysorbate 20. Reconstitution of the single-use vial with 1.3 ml sterile water for injection yields approximately 1.5 ml solution to deliver 125 mg per 1.25 ml (100 mg/ml) of antibody.

(c) A sterile, preservative-free lyophilized powder for intravenous (i.v.) administration. The product can be formulated with α,α-trehalose dihydrate, L-histidine HCl, histidine and polysorbate 20 USP. For example, each vial can contain 440 mg anti-CD19 antibody, 400 mg α,α-trehalose dihydrate, 9.9 mg L-histidine HCl, 6.4 mg L-histidine, and 1.8 mg polysorbate 20, USP. Reconstitution with 20 ml of bacteriostatic water for injection (BWFI), USP, containing 1.1% benzyl alcohol as a preservative, yields a multi-dose solution containing 21 mg/ml antibody at a pH of approximately 6.

(d) A sterile, lyophilized powder for intravenous infusion in which an anti-CD 19 antibody is formulated with sucrose, polysorbate, monobasic sodium phosphate monohydrate, and dibasic sodium phosphate dihydrate. For example, each single-use vial can contain 100 mg anti-CD19 antibody, 500 mg sucrose, 0.5 mg polysorbate 80, 2.2 mg monobasic sodium phosphate monohydrate, and 6.1 mg dibasic sodium phosphate dihydrate. No preservatives are present. Following reconstitution with 10 ml sterile water for injection, USP, the resulting pH is approximately 7.2.

(e) A sterile, preservative-free solution for subcutaneous administration supplied in a single-use, 1 ml pre-filled syringe or auto-injector. The product can be formulated with sodium chloride, monobasic sodium phosphate dihydrate, dibasic sodium phosphate dihydrate, sodium citrate, citric acid monohydrate, mannitol, polysorbate 80 and water for injection, USP. Sodium hydroxide may be added to adjust pH to about 5.2.

For example, each syringe can be formulated to deliver 0.8 ml (40 mg) of drug product. Each 0.8 ml contains 40 mg anti-CD19 antibody, 4.93 mg sodium chloride, 0.69 mg monobasic sodium phosphate dihydrate, 1.22 mg dibasic sodium phosphate dihydrate, 0.24 mg sodium citrate, 1.04 citric acid monohydrate, 9.6 mg mannitol, 0.8 mg polysorbate 80 and water for injection, USP.

(f) A sterile, preservative-free, lyophilized powder contained in a single-use vial that is reconstituted with sterile water for injection (SWFI), USP, and administered as a subcutaneous (s.c.) injection. The product can be formulated with sucrose, histidine hydrochloride monohydrate, L-histidine, and polysorbate. For example, a 75 mg vial can contain 129.6 mg or 112.5 mg of an anti-CD19 antibody, 93.1 mg sucrose, 1.8 mg L-histidine hydrochloride monohydrate, 1.2 mg L-histidine, and 0.3 mg polysorbate 20, and is designed to deliver 75 mg of the antibody in 0.6 ml after reconstitution with 0.9 ml SWFI, USP. A 150 mg vial can contain 202.5 mg or 175 mg anti-CD19 antibody, 145.5 mg sucrose, 2.8 mg L-histidine hydrochloride monohydrate, 1.8 mg L-histidine, and 0.5 mg polysorbate 20, and is designed to deliver 150 mg of the antibody in 1.2 ml after reconstitution with 1.4 ml SWFI, USP.

(g) A sterile, hyophilized product for reconstitution with sterile water for injection. The product can be formulated as single-use vials for intramuscular (IM) injection using mannitol, histidine and glycine. For example, each single-use vial can contain 100 mg anti-CD 19 antibody, 67.5 mg of mannitol, 8.7 mg histidine and 0.3 mg glycine, and is designed to deliver 100 mg antibody in 1.0 ml when reconstituted with 1.0 ml sterile water for injection. As another example, each single-use vial can contain 50 mg anti-CD19 antibody, 40.5 mg mannitol, 5.2 mg histidine and 0.2 mg glycine, and is designed to deliver 50 mg of antibody when reconstituted with 0.6 ml sterile water for injection.

(h) A sterile, preservative-free solution for intramuscular (IM) injection, supplied at a concentration of 100 mg/ml. The product can be formulated in single-use vials with histidine, glycine, and sterile water for injection. For example, each single-use vial can be formulated with 100 mg anti-CD19 antibody, 4.7 mg histidine, and 0.1 mg glycine in a volume of 1.2 ml designed to deliver 100 mg of antibody in 1 ml. As another example, each single-use vial can be formulated with 50 mg antibody, 2.7 mg histidine and 0.08 mg glycine in a volume of 0.7 ml or 0.5 ml designed to deliver 50 mg of antibody in 0.5 ml.

### Dosing

Those skilled in the art will appreciate that dosages and treatment regimens can be selected based on a number of factors including the age, sex, race and disease condition of the subject (e.g., the stage and/or form of MS). For example, appropriate dosage and treatment regimens can be determined by one of skill in the art for particular stages and/or forms of MS in a patient or patient population. Dose response curves can be generated using standard protocols in the art in order to determine the effective amount of compositions of the disclosure for treating patients having different stages and/or forms of MS. For example, effective amounts of compositions of the disclosure may be extrapolated from dose-response curves derived in vitro test systems or from animal model (e.g., the cotton rat or monkey) test systems. Models and methods for evaluation of the effects of antibodies are known in the art (Wooldridge et al., Blood, 89(8): 2994-2998 (1997)), incorporated by reference herein in its entirety). In general, patients having more advanced stages and/or more severe forms of MS will require higher doses and/or more frequent doses which may be administered over longer periods of time in comparison to patients having less advanced stages and/or forms of MS. In certain embodiments, treatment regimens standard in the art for antibody therapy can be used with compositions and methods of the disclosure.

CD19 density measurements (e.g., the density of CD19 on the surface of the patient's B cells) may also help determine a subject's appropriate treatment regimen and dosage. Methods of determining the density of antibody binding to cells are known to those skilled in the art (See, e.g., Sato et al., J. Immunology 165:6635-6643 (2000); which discloses a method of assessing cell surface density of specific CD antigens). Other standard methods include Scatchard analysis. For example, the antibody or fragment can be isolated, radiolabeled, and the specific activity of the radiolabeled antibody determined. The antibody is then contacted with a target cell expressing CD19. The radioactivity associated with the cell can be measured and, based on the specific activity, the amount of antibody or antibody fragment bound to the cell determined.

Another suitable method to assay for CD19 density employs fluorescence activated flow cytometry. Generally, the antibody or antibody fragment is bound to a target cell expressing CD19. A second reagent that binds to the antibody is then added, for example, a fluorochrome labeled anti-immunoglobulin antibody. Fluorochrome staining can then be measured and used to determine the density of antibody or antibody fragment binding to the cell.

In certain embodiments, the dose of a composition comprising anti-CD19 antibody is measured in units of mg/kg of patient body weight. In other embodiments, the dose of a composition comprising anti-CD19 antibody is measured in units of mg/kg of patient lean body weight (i.e., body weight minus body fat content). In yet other embodiments, the dose of a composition comprising anti-CD 19 antibody is measured in units of mg/m² of patient body surface area. In yet other embodiments, the dose of a composition comprising anti-CD19 antibody is measured in units of mg per dose administered to a patient. Any measurement of dose can be used in conjunction with compositions and methods of the present disclosure and dosage units can be converted by means standard in the art.

In some embodiments of the present disclosure, anti-CD19 antibodies bind to B cells and may result in efficient (i.e., at low dosage) depletion of B cells (as described herein). Higher degrees of binding may be achieved where the density of human CD19 on the surface of a patient's B cells is high. In certain embodiments, dosages of the antibody (optionally in a pharmaceutically acceptable carrier as part of a pharmaceutical composition) are at least about 0.0005, 0.001, 0.05, 0.075, 0.1, 0.25, 0.375, 0.5, 1, 2.5, 5, 10, 20, 37.5, or 50 mg/m² and/or less than about 500, 475, 450, 425, 400, 375, 350, 325, 300, 275, 250, 225, 200, 175, 150, 125, 100, 75, 60, 50, 37.5, 20, 15, 10, 5, 2.5, 1, 0.5, 0.375, 0.1, 0.075 or 0.01 mg/m². In certain embodiments, the dosage is between about 0.0005 to about 200 mg/m², between about 0.001 and 150 mg/m², between about 0.075 and 125 mg/m², between about 0.375 and 100 mg/m², between about 2.5 and 75 mg/m², between about 10 and 75 mg/m², and between about 20 and 50 mg/m². In related embodiments, the dosage of anti-CD19 antibody used is at least about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5 mg/kg of body weight of a patient. In certain embodiments, the dose of anti-CD19 antibody used is at least about 0.1 to 1, 1 to 5, 1 to 10, 5 to 15, 10 to 20, or 15 to 25 mg/kg of body weight of a patient. In certain embodiments, the dose of anti-CD19 antibody used is at least about 0.1 to 1, 1 to 5, 1 to 20, 3 to 15, or 5 to 10 mg/kg of body weight of a patient. In other embodiments, the dose of anti-CD19 antibody used is at least about 5, 6, 7, 8, 9, or 10 mg/kg of body weight of a patient. In certain embodiments, a single dosage unit of the antibody (optionally in a pharmaceutically acceptable carrier as part of a pharmaceutical composition) can be at least about 0.5, 1, 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 204, 206, 208, 210, 212, 214, 216, 218, 220, 222, 224, 226, 228, 230, 232, 234, 236, 238, 240, 242, 244, 246, 248, or 250 mg/m². In other embodiments, dosage of anti-CD19 antibody is up to 1 g per single dosage unit. In other embodiments, the dosage of anti-CD19 antibody ranges from 10 mg to 1000 mg per single dosage unit. In some embodiments, the dosage of anti-CD19 antibody ranges from 10 mg to 100 mg, 15 mg to 150 mg, 100 mg to 200 mg, 150 mg to 250 mg, 200 mg to 300 mg, 250 mg to 350 mg, 300 mg to 400 mg, 350 mg to 450 mg, 400 mg to 500 mg, 450 mg to 550 mg, 500 mg to 600 mg, 550 mg to 650 mg, 600 mg to 700 mg, 650 mg to 750 mg, 700 mg to 800 mg, 750 mg to 850 mg, 800 mg to 900 mg, 850 mg to 950 mg, or 900 mg to 1000 mg per single dosage unit.

All of the above doses are exemplary and can be used in conjunction with compositions and methods of the present disclosure. However where an anti-CD19 antibody is used in conjunction with a toxin or radiotherapeutic agent the lower doses described above may be preferred. In certain embodiments, where the patient has low levels of CD19 density, the lower doses described above may be preferred.

In some embodiments of methods of this present disclosure, antibodies and/or compositions of this present disclosure can be administered at a dose lower than about 375 mg/m²; at a dose lower than about 37.5 mg/m²; at a dose lower than about 0.375 mg/m²; and/or at a dose between about 0.075 mg/m² and about 125 mg/m². In certain embodiments of methods of the present disclosure, dosage regimens comprise low doses, administered at repeated intervals. For example, in one embodiment, compositions of the present disclosure can be administered at a dose lower than about 375 mg/m² at intervals of approximately every 1, 2, 3, 4, 5, 6, 7, 8, 9, 10, 15, 20, 25, 30, 35, 40, 45, 50, 60, 70, 80, 90, 100, 125, 150, 175, or 200 days.

The specified dosage can result in B cell depletion in the human treated using compositions and methods of the present disclosure for a period of at least about 1, 2, 3, 5, 7, 10, 14, 20, 30, 45, 60, 75, 90, 120, 150 or 180 days or longer. In certain embodiments, pre-B cells (not expressing surface immunoglobulin) are depleted. In certain embodiments, mature B cells (expressing surface immunoglobulin) are depleted. In other embodiments, all non-malignant types of B cells can exhibit depletion. Any of these types of B cells can be used to measure B cell depletion. B cell depletion can be measured in bodily fluids such as blood serum, or in tissues such as bone marrow. In certain embodiments of methods of the present disclosure, B cells are depleted by at least 30%, 40%, 50%, 60%, 70%, 80%, 90%, or 100% in comparison to B cell levels in the patient being treated before use of compositions and methods of the present disclosure. In other embodiments of methods of the present disclosure, B cells are depleted by at least 30%, 40%, 50%, 60%, 70%, 80%, 90%, or 100% in comparison to typical standard B cell levels for humans. In related embodiments, the typical standard B cell levels for humans are determined using patients comparable to the patient being treated with respect to age, sex, weight, and other factors.

In certain embodiments of the present disclosure, the dose can be escalated or reduced to maintain a constant dose in the blood or in a tissue, such as, but not limited to, bone marrow. In related embodiments, the dose is escalated or reduced by about 2%, 5%, 8%, 10%, 15%, 20%, 30%, 40%, 50%, 60%, 70%, 80%, 90%, and 95% in order to maintain a desired level of an antibody of compositions and methods of the present disclosure.

In certain embodiments, the dosage can be adjusted and/or the infusion rate can be reduced based on patient's immunogenic response to compositions and methods of the present disclosure.

According to certain embodiments, a dosage protocol (e.g., treatment regimen) comprises administering an effective amount of a CD19 antibody to the MS subject to provide an initial antibody exposure of 0.1 to 1, 1 to 5, 1 to 10, 5 to 15, 10 to 20, or 15 to 25 mg/kg of body weight of a patient followed by a second CD19 antibody exposure of 0.1 to 1, 1 to 5, 1 to 10, 5 to 15, 10 to 20, or 15 to 25 mg/kg of body weight of a patient, the second CD19 antibody exposure not being provided until from about 15 to 60 weeks from the initial antibody exposure. In some embodiments, a dosage protocol comprises administering an initial antibody exposure of 10 mg to 1000 mg, 10 mg to 200 mg, 100 mg to 300 mg, 200 mg to 400 mg, 300 mg to 500 mg, 400 mg to 600 mg, 500 mg to 700 mg, 600 mg to 800 mg, 700 mg to 900 mg, or 800 mg to 1000 mg of a CD19 antibody to the MS subject, followed by a second antibody exposure of 10 mg to 1000 mg, 10 mg to 200 mg, 100 mg to 300 mg, 200 mg to 400 mg, 300 mg to 500 mg, 400 mg to 600 mg, 500 mg to 700 mg, 600 mg to 800 mg, 700 mg to 900 mg, or 800 mg to 1000 mg of a CD19 antibody, the second CD19 antibody exposure not being provided until from about 15 to 60 weeks from the initial antibody exposure. In some embodiments, the second exposure is not administered until from about 15-20 weeks, from about 17-23 weeks, from about 20-25 weeks, from about 23-27 weeks, from about 25-30 weeks, from about 27-33 weeks, from about 30-35 weeks, from about 33-37 weeks, from about 35-40 weeks, from about 37-43 weeks, from about 40-45 weeks, from about 43-47 weeks, from about 45-50 weeks, from about 47-53 weeks, from about 50-55 weeks, from about 53-57 weeks, or from about 55-60 weeks from the initial exposure. For purposes of this disclosure, the second CD19 antibody exposure is the next time the subject is treated with the CD19 antibody after the initial antibody exposure, there being no intervening CD19 antibody treatment or exposure between the initial and second exposures.

In some embodiments, the second CD 19 antibody exposure is not provided until about 20 to 30 weeks from the initial exposure, optionally followed by a third CD19 antibody exposure of about 1 to 10, 5 to 15, 10 to 20, or 15 to 25 mg/kg of body weight of a patient. In some embodiments, the third CD19 antibody exposure comprises 10 mg to 1000 mg, 10 mg to 200 mg, 100 mg to 300 mg, 200 mg to 400 mg, 300 mg to 500 mg, 400 mg to 600 mg, 500 mg to 700 mg, 600 mg to 800 mg, 700 mg to 900 mg, or 800 mg to 1000 mg. In embodiments where a third exposure is administered, the third exposure is not administered until from about 40 to 60 weeks, or from about 40 to 46 weeks, or from about 43-47 weeks, or from about 45-50 weeks, or from about 47-53 weeks, or from about 50-55 weeks, or from about 53-57 weeks, or from about 55-60 weeks from the initial exposure. In certain embodiments, no further CD19 antibody exposure is provided until at least about 70-75 weeks from the initial exposure.

Any one or more of the antibody exposures described herein may be provided to the patient as a single dose of antibody, or as two separate doses of the antibody (i.e., constituting a first and second dose). The particular number of doses (whether one or two) employed for each antibody exposure is dependent, for example, on the type of MS treated, the type of antibody employed, whether and what type of second medicament is employed, and the method and frequency of administration. Where two separate doses are administered, the second dose is preferably administered from about 3 to 17 days, from about 6 to 16 days, from about 13 to 16 days, or 15 days from the time the first dose was administered. Where two separate doses are administered, the first and second dose of the antibody is preferably about 1 to 10, 5 to 15, 10 to 20, or 15 to 25 mg/kg of body weight of a patient. In some embodiments, the first and second doses comprise 10 mg to 1000 mg, 10 mg to 200 mg, 100 mg to 300 mg, 200 mg to 400 mg, 300 mg to 500 mg, 400 mg to 600 mg, 500 mg to 700 mg, 600 mg to 800 mg, 700 mg to 900 mg, or 800 mg to 1000 mg of a CD19 antibody to the MS subject.

### Articles of Manufacture

In certain embodiments, an article of manufacture containing materials useful for the treatment of multiple sclerosis described above is provided. In some embodiments, the article of manufacture comprises: (a) a container comprising a composition comprising an antibody that binds to CD19 and a pharmaceutically acceptable carrier or diluent within the container; and (b) a package insert with instructions for administering the composition to a subject suffering from multiple sclerosis to provide an initial antibody exposure of about 1 to 10, 5 to 15, 10 to 20, or 15 to 25 mg/kg of body weight of the subject followed by at least a second antibody exposure of about 1 to 10, 5 to 15, 10 to 20, or 15 to 25 mg/kg of body weight of the subject, the at least second exposure not being provided until from about 16 to 60 weeks from the initial exposure.

The article of manufacture comprises a container and a label or package insert on or associated with the container. Suitable containers include, for example, bottles, vials, syringes, etc. The containers may be formed from a variety of materials such as glass or plastic. The container holds or contains a composition that is effective for treating the multiple sclerosis and may have a sterile access port (for example the container may be an intravenous solution bag or a vial having a stopper pierceable by a hypodermic injection needle). At least one active agent in the composition is the antibody. The label or package insert indicates that the composition is used for treating multiple sclerosis in a subject suffering therefrom with specific guidance regarding dosing amounts and intervals of antibody and any other drug being provided. The article of manufacture may further comprise a second container comprising a pharmaceutically acceptable diluent buffer, such as bacteriostatic water for injection, phosphate-buffered saline, Ringer's solution and dextrose solution. The article of manufacture may further include other materials desirable from a commercial and user standpoint, including other buffers, diluents, filters, needles, and syringes.

### EXAMPLES

### Example 1

### The 300B4-CD19 Binding Assay

The ability of chimeric, humanized, or human anti-CD19 antibodies to bind hCD19 can be assessed in a cell based CD19 binding assay utilizing 300B4 cells expressing recombinant cell-surface human CD19 as a capture agent. 300B4 cells are cultured according to standard protocols in RPMI 1640 medium containing L-glutamine and supplemented with 10% Fetal Calf Serum, β-mercaptoethanol in the presence of 1 mg/ml G418. A standard ELISA protocol can be used for the cell based CD19 binding assay. For example, individual wells of a 96 well U bottom plate are seeded with 1×10⁵ 300 B4 cells and incubated overnight. Cells are washed once with ELISA buffer prior to incubation on ice with human, humanized, or chimeric anti-CD19 antibodies. Binding reactions are performed in triplicates for each antibody concentration tested. Negative control wells using an isotype matched antibody of irrelevant specificity should be included in the assay. Following incubation with the antibody 300B4 cells are washed three times with 200 micro liter of ELISA buffer. The amount of chimeric, humanized, or human anti-CD19 antibodies bound to 300B4 cells can be detected using a goat anti-human kappa antibody conjugated with horseradish peroxidase according to standard protocols.

### Example 2

### In Vitro ADCC Assay

The CytoTox 96™ Non-Radioactive Cytotoxicity Assay (Promega) is a calorimetric alternative to ⁵¹Cr release cytotoxicity assays. The CytoTox 96™ Assay quantitatively measures lactate dehydrogenase (LDH), a stable cytosolic enzyme that is released upon cell lysis. Released LDH in culture supernatants is measured with a 30-minute coupled enzymatic assay, which results in the conversion of a tetrazolium salt (INT) into a red formazan product. The amount of color formed is proportional to the number of lysed cells.

The assays are performed according to the manufacturer's directions. Briefly, target cells are washed with PBS, resuspended in RPMI-5 Phenol Free media at a cell density of 0.4×10⁶/ml. NK effector cells are washed once in PBS and resuspended in RPMI-5 Phenol Free media at a cell density 1×10⁶/ml. Assays are performed in U bottom 96 well plates. Each assay plate includes a combination of experimental and control wells. Experimental wells are set up by combining 50 μl of the appropriate antibody dilution, 50 μl of target cell suspension and 50 μl of effector cell suspension. The cell densities described above result in a 1:2.5 target to effector cell ratio; effector cell stock may be further diluted or concentrated if a different target to effector ratio is desired. Several different types of control wells are used to account for (i) the spontaneous LDH release form target cells (Target Spontaneous), (ii) the spontaneous LDH release from effector cells (Effector Spontaneous), (iii) the maximum LDH release from the target cells (Target Maximum), and (iv) the presence of contaminants in the culture medium (Background). All wells in use on a 96 well plate contain the same final volume. Reactions are set up in triplicates. Following set up, plates are spun at 120×g for 3 minutes to pellet the cells. Incubate plate at 37° C. and 5% CO₂ for 4 hours. Forty five minutes prior to the end of incubation 15 μl of manufacturer provided Lysis Buffer is added to the Target Cell Maximum Release Control well. After incubation the plate is centrifuged at 120×g for 4 minutes. 50 μl of the supernatant from each well is transferred to a new flat bottom 96 well plate. 50 μl of reconstituted substrate mix (assembled from manufacturer provided components) is added and the plate is incubated at room temperature 10-20 minutes protected from light. 50 μl of manufacturer provided stop buffer is added and absorbance at 490 or 492 nm is measured in a plate reader. % cytotoxicity equals (Experimental−Effector spontaneous−Target Spontaneous)/(Target Maximum−Target Spontaneous). Prior to calculating the % cytotoxicity all other values are reduced by the Background.

### Example 3

### In Vitro FcγRIIIA Receptor Binding Assay

Relative binding affinity of various humanized anti-CD19 antibody preparations to human FcγRIIIA receptor (CD16) may be ascertained using an ELISA assay. Microtiter plates are coated with 50 μl antibody preparation (50 μg/ml) at 4° C. overnight. Any remaining binding sites are blocked with 4% skimmed milk in PBS buffer (blocking buffer) for 1 h at 37° C. After washing the wells, 50 μl of serially diluted monomeric FcγRIIIA-flag protein is added to each well and incubated for 60 min at 37° C. 50 μl of 2.5 μg/ml anti-flag-ME-biotin (Sigma) is added to each well and incubated for 30 min at 37° C. Wells are washed between incubation with each of the following reagents. 50 μl of 0.1 μg/ml avidin-conjugated HRP (PIERCE) is added to each well and incubated for 30 min at 37° C. Detection is carried out by adding 30 μl of tetramethylbenzidine (TMB) substrate (Pierce) followed by neutralization with 30 μl of 0.2 M H₂SO₄. The absorbance is read at 450 nm.

Relative binding affinity of various humanized anti-CD19 antibody preparations to various human and murine FcγRs may also be ascertained on a BIAcore 3000 instrument (BIAcore AB, Uppsala, Sweden). In brief, the fucosylated and afucosylated forms of the IgG1-humanized CD19 mAb were immobilized onto separate flow cells on a CM5 sensor chips using a standard amino coupling chemistry as outlined by the instrument's manufacturer. A reference flow cell without mAb was also prepared on each sensor chip. Stock solutions of FcγRs (Oganesvan et al., 2008) were serially diluted using instrument buffer (HBS-EP buffer containing 0.01 M HEPES, pH 7.4, 0.15 M NaCl, 3 mM EDTA, and 0.005% P-20 detergent). FcγRs were injected over both the IgG and reference cell surfaces at a flow rate of 5 μl/min. Binding data were collected for 50 min, followed by a 60-se pulse of 5 mM HCl between injections to remove bound FcγR from the IgG surfaces. After all binding data were collected, individual data sets were averaged for binding (response at equilibrium) at each concentration, and then fit to a 1:1 binding isotherm (response at equilibrium versus concentration) plot. From this, the K_{D} values were derived. Such calculations were performed with BIAevaluation software, version 4.1 (BIAcore AB).

### Example 4

### Expression of CD19 on Plasma Cells from Human CD19 Transgenic (huCD19Tg) Mice

In the BLIMP gfp mice, plasma cells are engineered to express green florescence protein (GFP), allowing easy identification of these cells. BLIMPgfp+huCDl9Tg mice were created by breeding and spleen and bone marrow cells from these mice were harvested and prepared into single cell suspensions for flow cytometry. Cells were stained with anti-CD45R/B220 (AlexaFluor 700), anti-murine CD19 (PerCPCy5.5), and anti-human CD19 (APC Cy7). In mice, plasma cells express murine CD19 on cell surface, as well as transgenic human CD19. The level of huCD 19 on plasma cells is slightly lower to the B220+ B cells in the spleen and bone marrow and the level of muCD19 is lower on plasma cells, too.

### Example 5

### Effect of 16C4-aFuc on Antibody Titres and Plasma Cell Numbers in Ovalbumin (Ova) Immunized Human CD19 Transgenic (huCD19Tg) Mice

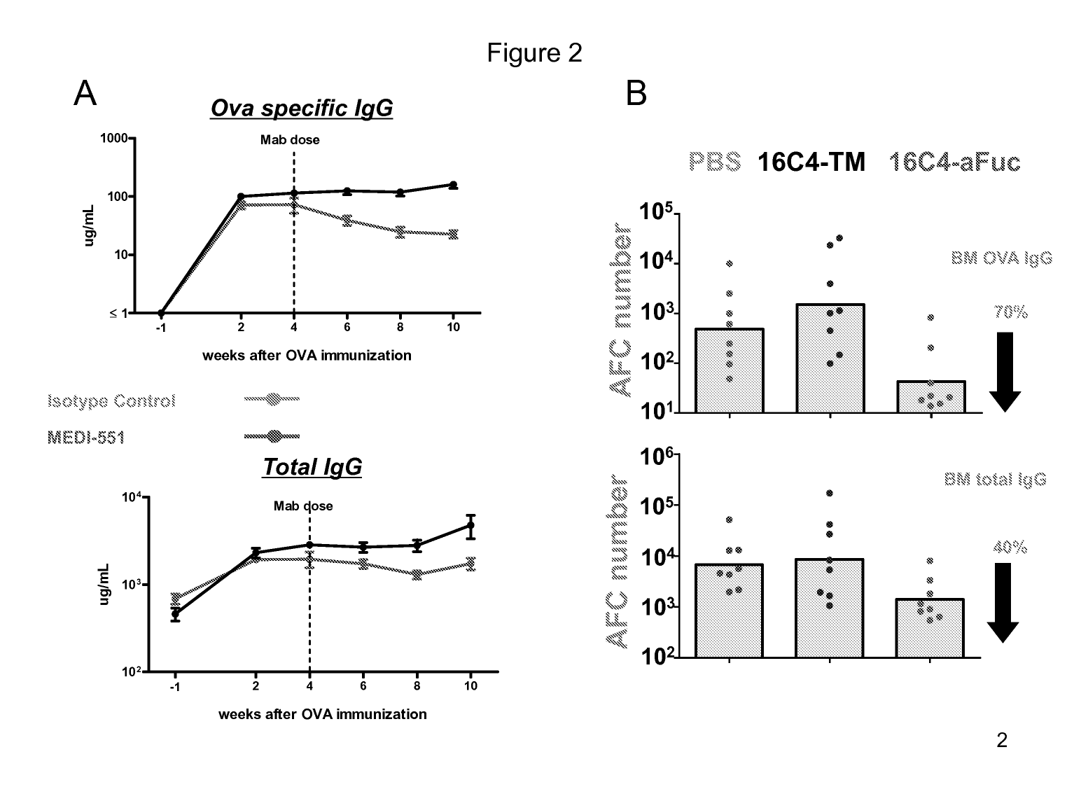

The effect of afucosylated MAb 16C4 (16C4-aFuc) on Ova-specific and total IgGwas examined. HuCD19Tg mice were immunized with 100 μg ovalbumin (ova) and CFA subcutaneously (s.c.). At four weeks following immunization, when the germinal centre reaction has subsided, mice were intravenously (IV) injected with 10 mg/kg 16C4-aFuc or isotype control (R347aFuc). Blood was collected retro-orbitally into heparanized tubes one week prior to immunization and every two weeks post immunization until the study endpoint at 10 weeks. Total IgG was determined by Luminex assay following the manufacturer's protocol for the mouse Ig isotyping kit (Millipore). Ova specific IgG were determined by ELISA. In brief, Ova was coated onto NUNC high binding plates at 4° C. overnight. Samples and standards (Santa Cruz) were incubated at RT for one hour following blockade. Plates were washed, then coated with goat anti-mouse total IgG HRP (Southern Biotech). Finally, OD values were determined after adding TMB substrate. The graphs in FIG. 2A show the mean+/−SE μg/mL of antibody.

After immunization all mice demonstrated 100-fold increased Ova specific IgG antibody titres and increased total IgG titres by week 2. Following treatment with 16C4-aFuc, total IgG and Ova specific IgG titre in serum are significantly reduced compared to mice treated with isotype control antibody.

The effect on IgG (total and Ova-specific) producing plasma cells from the bone marrow was examined: C57B1/6xhCD19Tg+/− mice were immunized with ova and CFA subcutaneously (s.c.). At four weeks following immunization mice were intravenously (IV) injected with 10 mg/kg 16C4-aFuc, the non-FcγReceptor binding control (16C4-TM), or PBS. Two weeks following MAb treatment, bone marrow (BM) cells were harvested from mice. Antibody forming cells (AFC) were detected by ELISpot, following the general manufacturer's protocol (MABTECH). Ova specific AFC were detected by coating plates with Ova then following the same general protocol. FIG. 2B shows that treatment with 16C4-aFuc significantly reduces the number of total IgG and Ova specific IgG AFC in the bone marrow, whereas, the non-FcγReceptor binding control (16C4TM) has no effect.

### Example 6

### Treatment of SLE1xhuCD19Tg Mice with 16C4-aFuc—Effect on Blood and Tissue B Cells

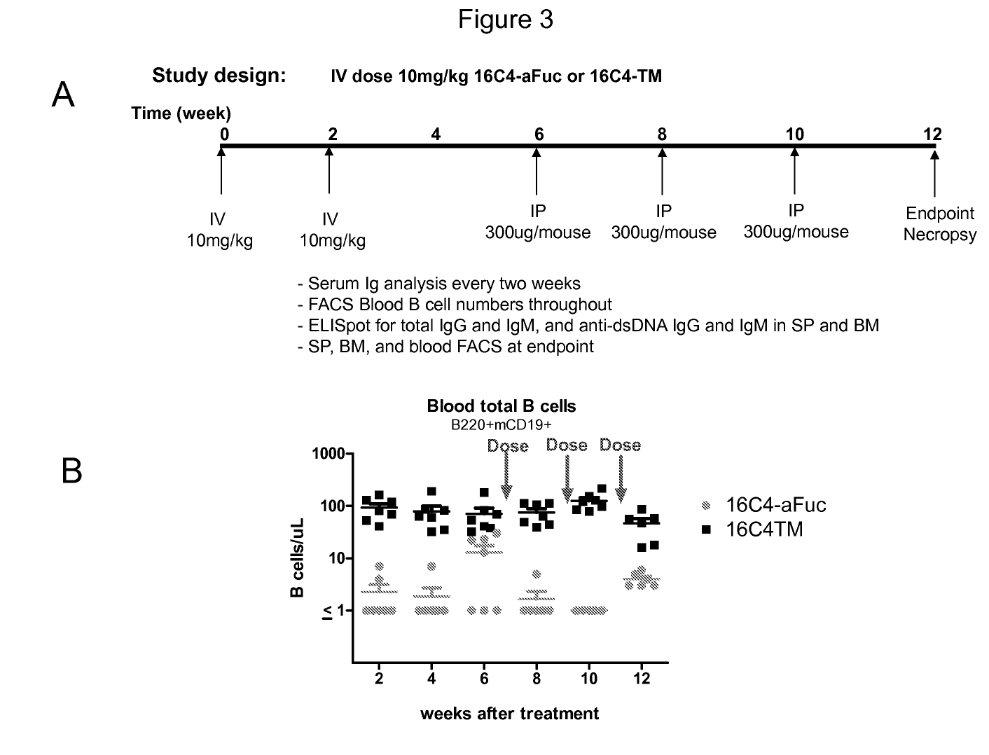

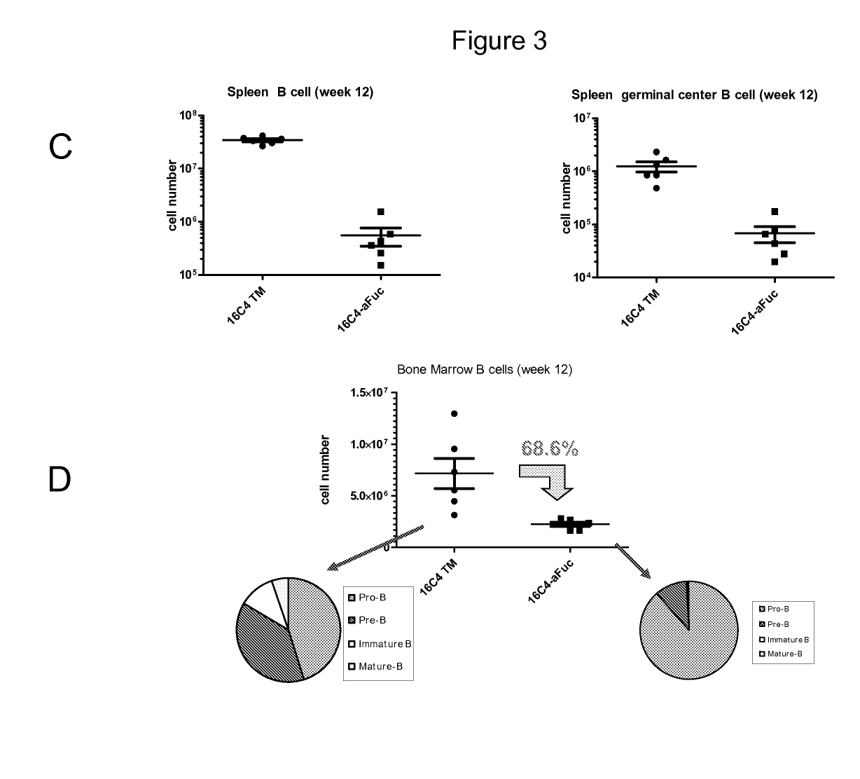

SLE1xhCD19Tg+/− mice were created by breeding SLE1+/+ mice with hCD19Tg mice, following MedImmune animal usage policy. At week 0, SLE1xhCD19Tg+/− mice were IV dosed either with 10 mg/kg 16C4-aFuc or the same dose of control antibody (16C4-TM). Mice received the same treatment at week 2 and subsequently IP injected with 16C4-aFuc at 300 μg/mouse for every two weeks starting at 6 weeks for up to 10 weeks. Blood was collected by retro-orbital bleeds into heparanized tubes every 2 weeks throughout the duration of the experiment. When the study was terminated at week 12, spleen, blood and bone marrow cells were recovered and subjected to FACS and ELISpot analysis. Serum samples were analyzed by ELISA. A schematic view of a longitudinal study of 16C4 in SLE1xhCD19Tg+/− mice is shown in FIG. 3A

Total circulating B cells were detected by FACS after staining with anti-CD45R/B220 and anti-murine CD19 MAb. The graph in FIG. 3B represents the number of B220+mCD19+ B cells in each mouse on the study, whether treated with 16C4-aFuc (orange circles) or the non-FcγReceptor binding control, 16C4-TM (black squares). All mice treated with 16C4-aFuc initially have >90% depletion of circulating B cells. A subset of mice had transient B cell recovery at 6 weeks following IV injection, but all mice once again are B cell depleted following IP injections until the end of the study.

The effect of 16C4-aFuc on total and germinal centre B cells in the spleen of SLE1xhuCD19Tg mice was examined at week twelve following multiple treatment with 16C4-aFuc. The SLExhCD19Tg mice have a >90% reduction of B cells in the spleen, compared to control MAb treated mice. The graphs in FIG. 3C depict the number of total B cells or germinal center B cells detected by FACS staining Total B cells were detected with anti-CD45R/B220 (AlexaFluor 700). Germinal center B cells were detected as PNA+ (FITC) and IgD− (Alexa 647).

The effect of 16C4-aFuc on B cell populations in the bone marrow of SLE1xhuCD19Tg mice was also examined. Bone marrow B cell numbers following treatment SLExhCD19Tg mice with 16C4-aFuc were determined by FACS staining Two femurs from the same mouse were collected at 12 weeks after study onset and single cell suspensions of cells were analyzed by FACS using the following antibodies: CD43 (FITC), IgM (PE-Cγ7), IgD (Alexa 647), CD45R/B220 (A700). Total B cell (B220+) numbers were reduced by 68% in 16C4 treated mice compared to control as shown in FIG. 3D. The majority of the B cells that remained were considered Pro-B cells (CD43+IgM−), which had very low expression of transgenic CD19.

### Example 7

### Treatment of SLE Mice with 16C4-aFuc—Effect on Spleen and Bone Marrow Plasma Cells

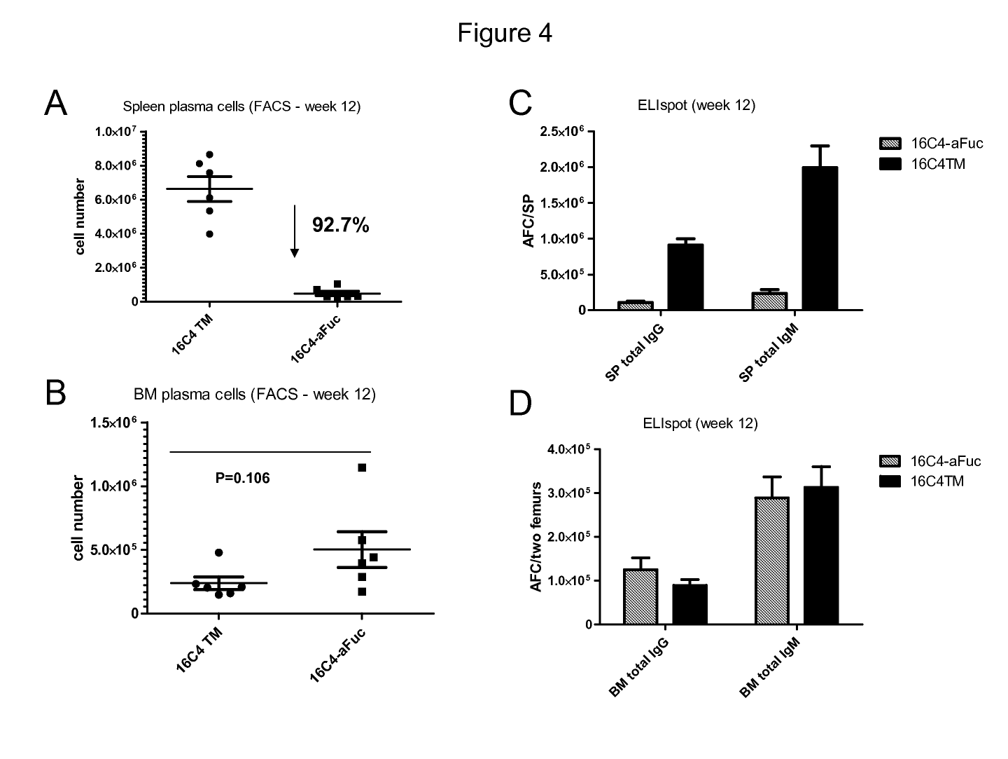

SLExhCD19Tg spleen and bone marrow plasma cells were detected by CD138 (PE) staining or by ELISpot following treatment with 16C4-aFuc. (see FIGS. 4A and 4B) FACS data is plotted as total number of CD138+ B cells. At 12 weeks, there is a 98% reduction of plasma cells in the spleen, whereas there is no significant change in plasma cell number in the bone marrow (BM). (see FIGS. 4C and 4D) ELISpot data for total IgG and IgM antibody forming cells (AFC) demonstrates a similar pattern, whereby there is a significant reduction in spleen AFC and no difference is detected in the BM.

### Example 8

### Treatment of SLE1xhuCD19Tg Mice with 16C4-aFuc—Effect on Anti-dsDNA Autoantibodies

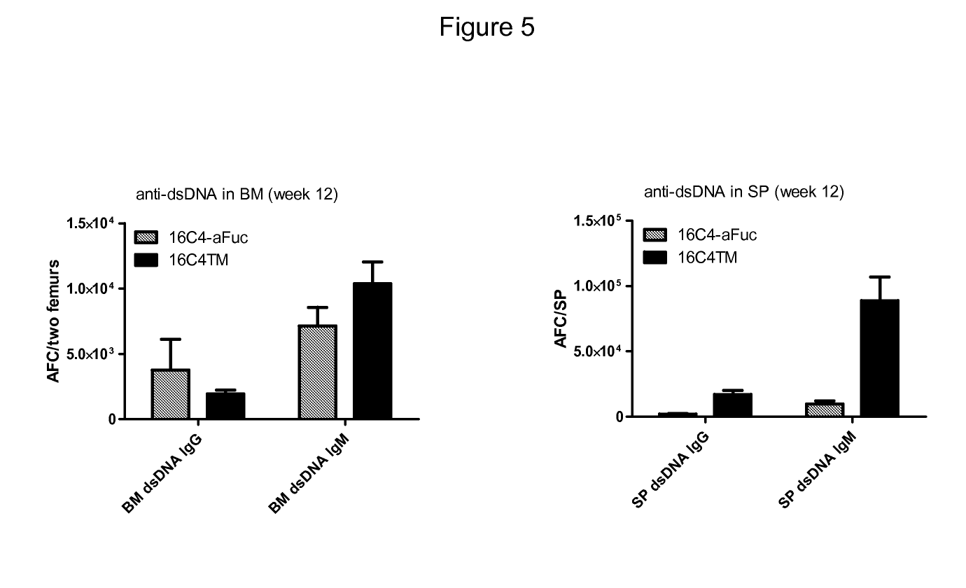

ELISpot assays were used to detect anti-dsDNA AFC in the spleen and BM of SLExhCD19Tg mice after 12 weeks of 16C4-aFuc treatment. Whereas there was no difference in the level of anti-dsDNA specific AFC in the BM, there was a significant reduction in spleen AFC specific for anti-dsDNA (see FIG. 5).

### Example 9

### Treatment of SLE1xhuCD19Tg Mice with 16C4-aFuc—Effect on Serum Immunoglobulins Over Time

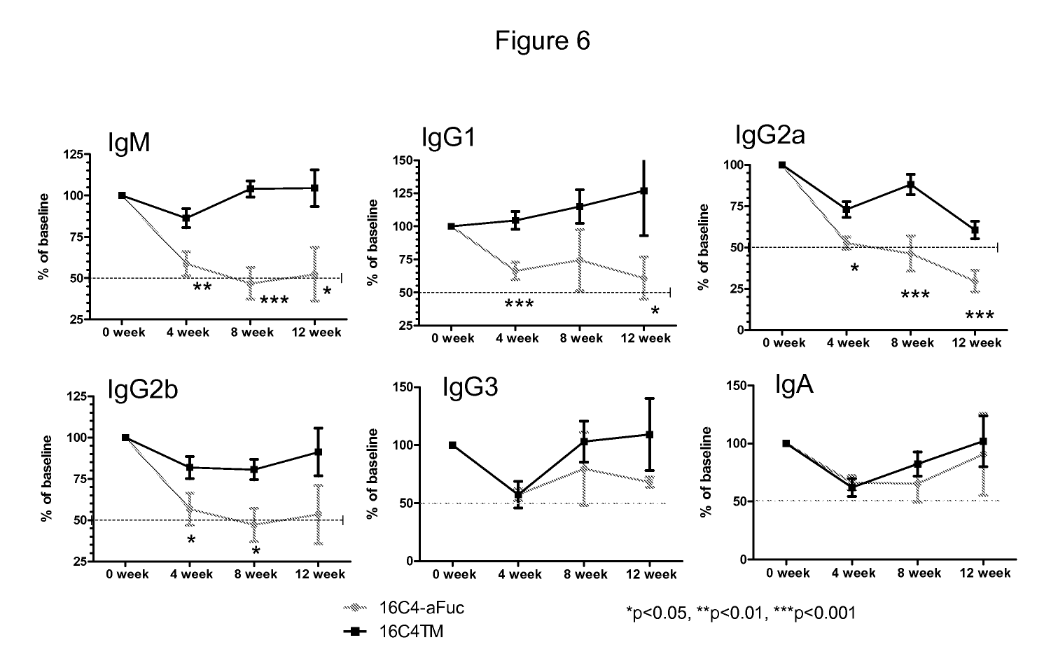

Serum was collected every two weeks from SLExhCD19Tg mice treated with 16C4-aFuc 2×/month for 12 weeks. Serum Ig levels were detected by luminex, following the manufacturer's protocol for the mouse Ig isotyping kit (MILLIPORE). FIG. 6 shows the resultant data plotted as a percentage of baseline (day 0) levels. There was a significant reduction in serum IgM, IgG1, IgG2a, and IgG2b in mice treated with 16C4-aFuc compared to the level before treatment. However the levels before and after isotype antibody treatment were similar. IgG3 and IgA were not significantly different.

### Example 10

### Treatment of SLE1xhuCD19Tg Mice with 16C4-aFuc—Reduction of Autoantibodies in Serum

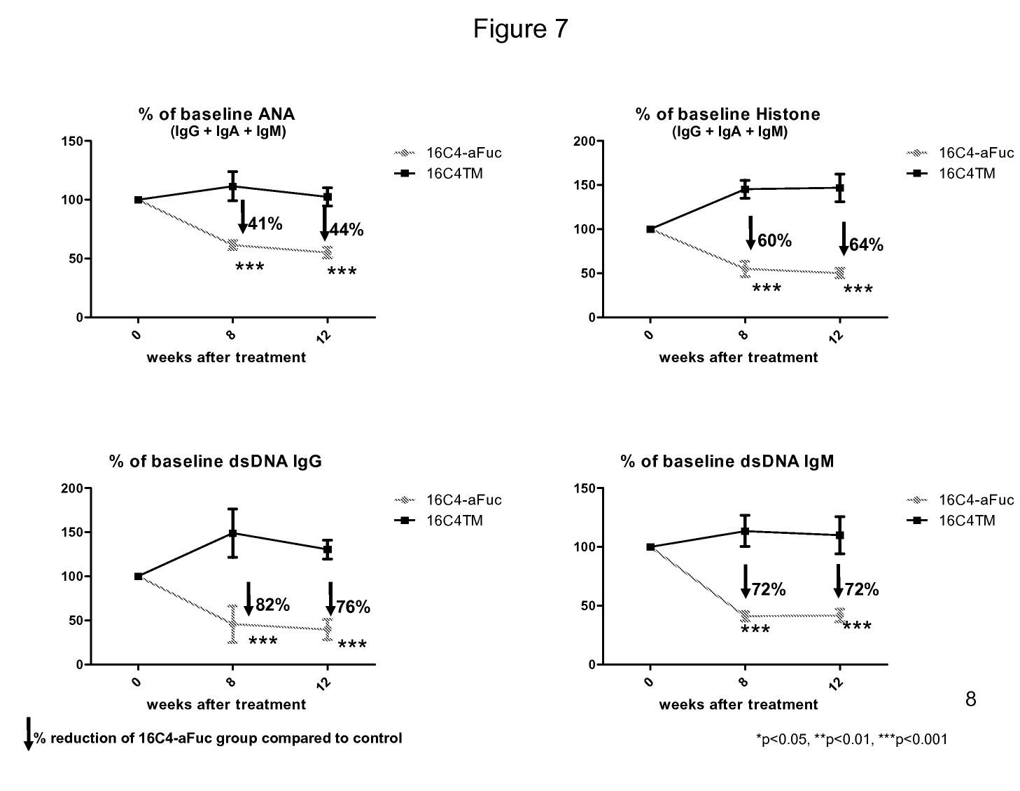

SLExhCD19Tg mice treated with 16C4-aFuc or the negative control MAb (16C4TM) for 12 weeks (dosed 2×/month). Autoantibody levels in the serum were detected using pre-made ELISA kits from Alpha Diagnostics (following the manufacturer's protocol). FIG. 7 shows the data for anti-ANA, anti-Histone and anti-dsDNA titres graphically as percentage of baseline (day 0). At 8 and 12 weeks there was a 40-80% reduction of autoantibody titres.

### Example 11

### CD19 Expression on Plasmablasts and Most Plasma Cells from Human Tissues

CD27+CD38high Cells Positive for CD19 have a Plasma Cell Phenotype and Secrete IgG

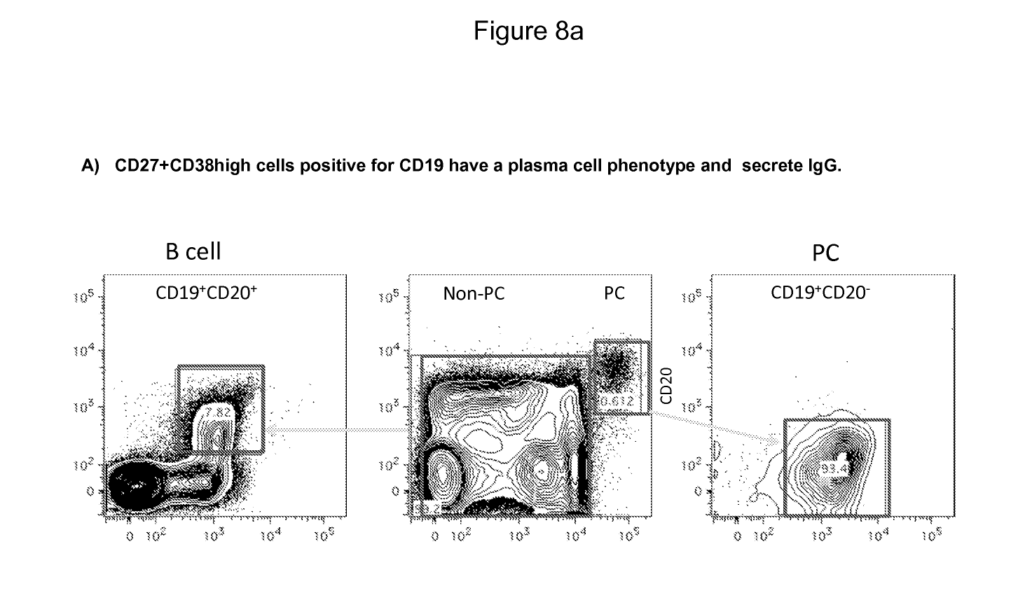

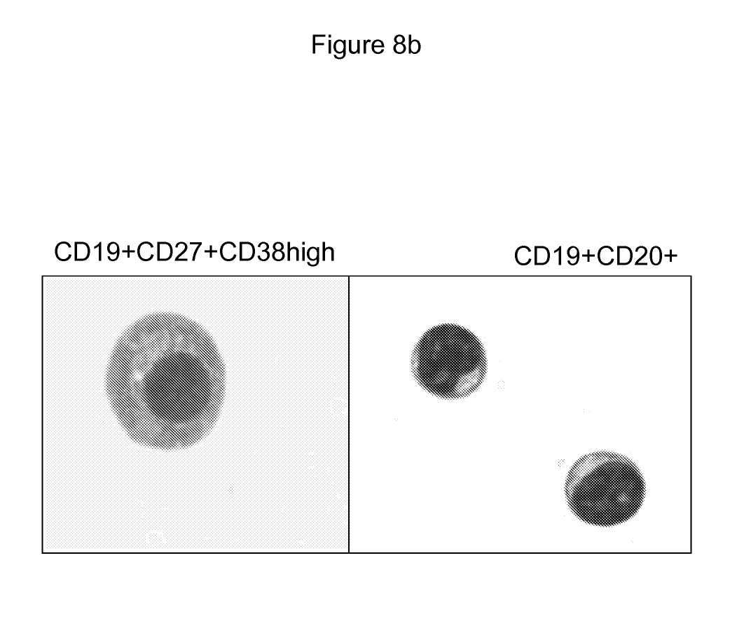

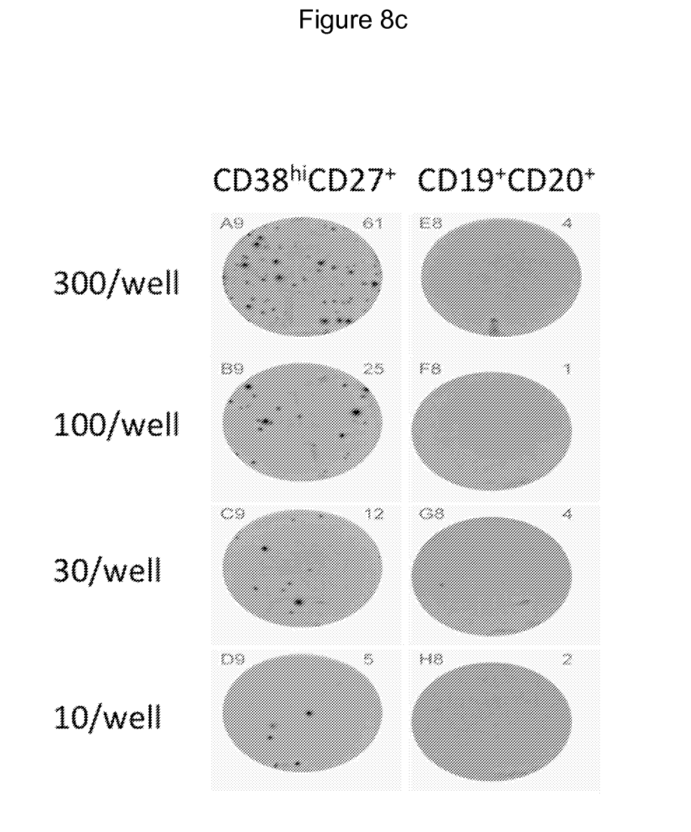

Bone marrow mononuclear ASC (CD38hiCD27+CD19+CD20−) and B cells (Non-PC gated, CD19+CD20+) were sorted via FCM on a BD FACSAria II cell sorter. Cells were separated into two fractions based on CD38 and CD27 expression. The CD38hiCD27+(PC) were further gated on CD19+CD20− and the Non-PC cells were further gated into CD19+CD20+ immature B cells (FACS panels, FIG. 8A). These 2 populations were sorted and further characterized by morphology (FIG. 8B). An example of Wright's stained sorted cells shows both fractions with a well defined large nucleus, however the CD19+PC sorted fraction exhibited characteristic perinuclear cytoplasm characteristic of PC which was not present in the CD19+CD20+Non-PC fraction.

Total IgG ELISpot of sorted cells shows the CD19+PC gated cells secrete IgG which was detectable at 10 cells/well (FIG. 8C). The CD19+CD20+Non-PC fraction did not show any real cell spots. From these results it can be seen CD27+CD38high cells positive for CD19 have a plasma cell phenotype and secrete IgG

CD19 is Expressed on CD27+CD38high Antibody Secreting Cells (ASC) from Blood and Lymphoid Organs

CD38highCD27+ cells from blood (PBMC) and lymphoid organs (tonsil, spleen, bone marrow) were analyzed for their relative expression of CD19 and CD20 by flow cytometry.

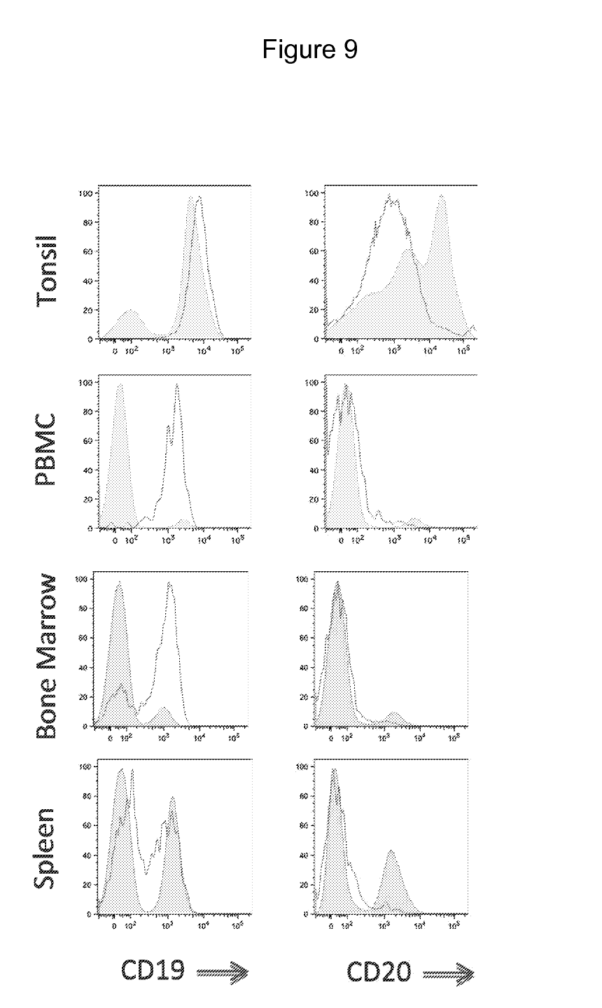

Representative overlays of CD19 (left column of FIG. 9) and CD20 (right column of FIG. 9) surface expression on CD38hiCD27+ ASC are shown as a solid line plot. Viable mononuclear cells for each tissue are shown for comparison under the shaded grey curve.

CD19 is expressed on most ASC from blood and the lymphoid tissues analyzed. Only the spleen and the bone marrow contain a distinct CD38highCD27+ population that is negative for CD19. In blood and most tissues analyzed CD38highCD27+ cells are negative for CD20.

A CD19 Neg. ASC Population from BM Contains Most of the Humoral Memory to Vaccine Antigens

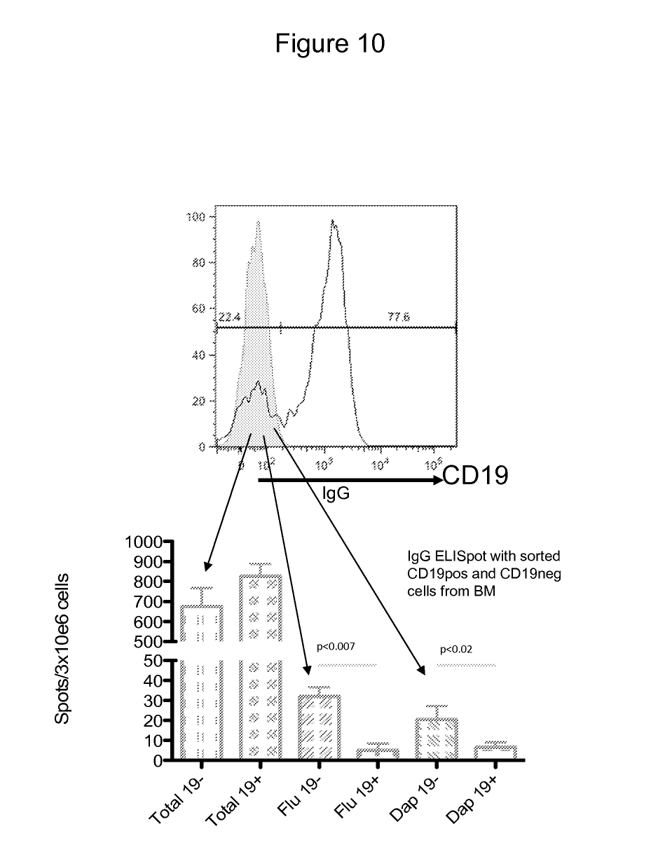

BM contains two distinct PC populations that can be differentiated based on their CD 19 expression. The majority of BM PC express CD19, while a minor population is CD19 negative (FACS panel, top of FIG. 10).

CD 19 positive and negative PC were analyzed for total IgG secretion and production of antibodies against specific vaccine antigens by ELIspot (bottom of FIG. 10). BM CD19− PC show memory-like specificity for vaccine antigens with increased frequency compared to CD19+ PC. CD19+ and CD19− show similar number of spots on total IgG ELISpot (500 sorted antibody secreting cells (ASC)). However, there is an increased number of ELISspots from ASC specific to Fluzone or Daptacel (3000 sorted ASC) among the CD19− fraction.

### Example 12

### Plasma Cell Signature

Using whole genome microarray analysis of sorted cellular fractions and purified PC from healthy volunteers, a signature score was developed combining expression levels of multiple PC enriched genes (IGHA, IGJ, IGKC, IGKV, and TNFRSF17) to estimate PC counts in patient samples. This newly developed gene expression based PC signature was used to monitor changes in this cell population in patients enrolled in MI-CP200, a Phase I dose escalation trial of 16C4afuc in scleroderma (NCT00946699). At various timepoints after a single dose of 16C4afuc (on days 3, 29, 85 in whole blood; day 29 in skin), gene expression changes in the PC and B cell signatures were evaluated in blood and skin samples. Changes in these signatures were calculated relative to each patient's baseline (day 1) sample.

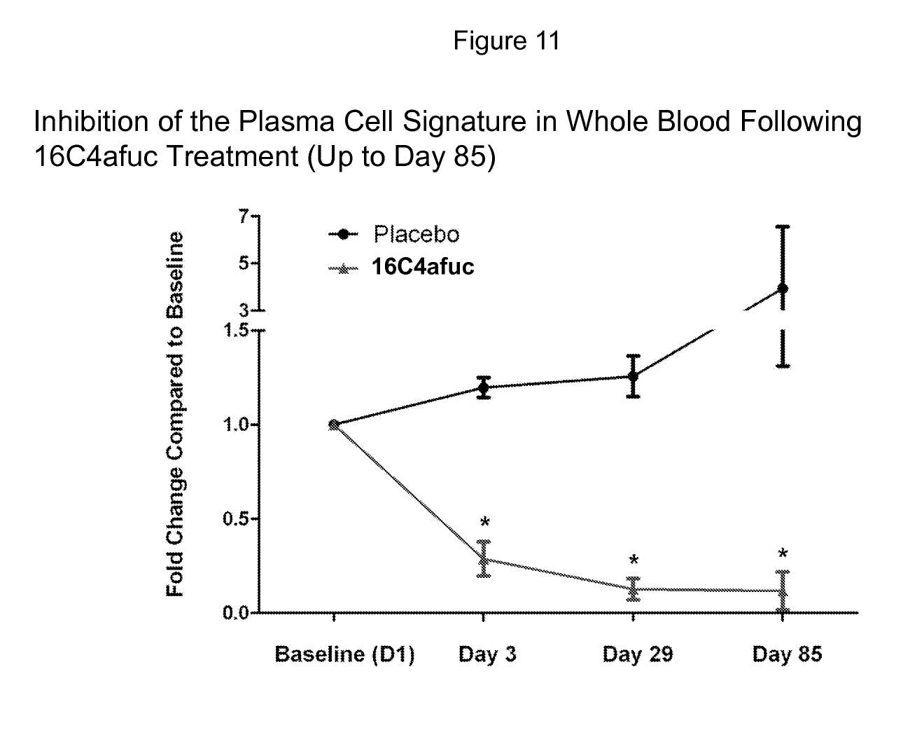

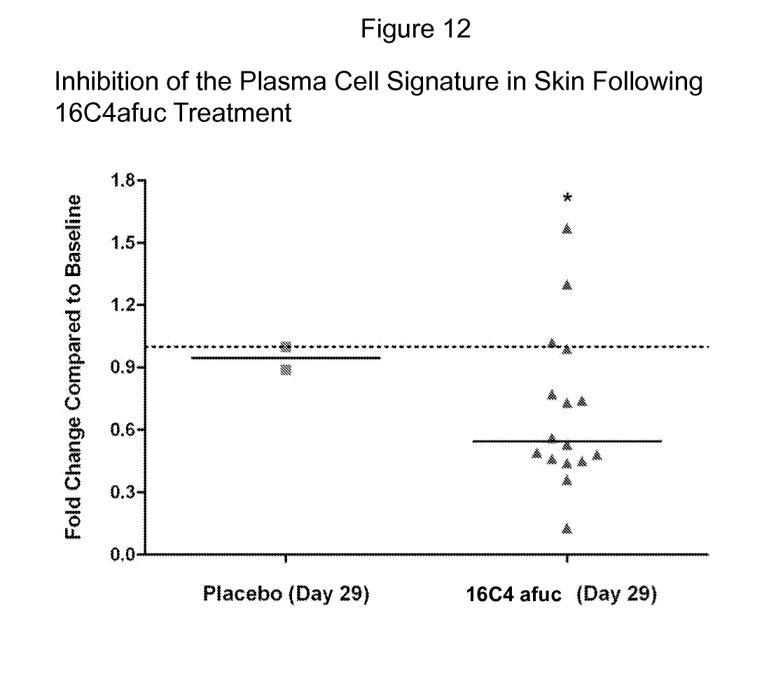

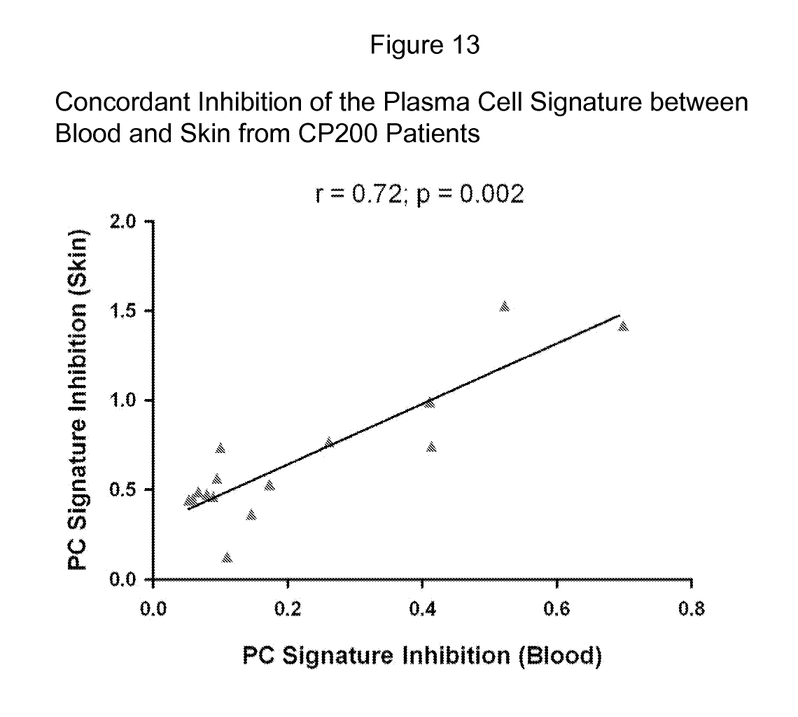

Results indicate that 16C4afuc caused a robust reduction of the PC gene signature in WB, with maximum depletion of approximately 98% and sustained depletion out to day 85 post-treatment (Figure A-1). By day 85, the last timepoint measured, the PC signature had recovered up to approximately 65% of baseline (FIG. 11). Differences between baseline and post MEDI-551 treatment values of the PC signature in WB were statistically significant (p<0.01) at all timepoints measured. A statistically significant reduction of the PC gene signature in skin samples following 16C4afuc treatment was also observed (p<0.05), reaching a maximum level of 90% and a median of approximately 55% (FIG. 12). Furthermore, in patients with matched blood and disease tissue specimens, there was concordant inhibition of the PC gene signature in WB and skin (Spearman rank correlation r=0.72, p=0.002; FIG. 13)

### Example 13

### Mediated Depletion of Human Plasma Cells (PC)

The capacity of 16C4afuc to mediate antibody dependent cell-mediated cytotoxic death of human plasma cells (CD27 high CD38 high) was evaluated.

For in vitro PC differentiation assays, fresh human blood was acquired from healthy donors (n=3) after receiving informed consent. Samples were drawn into CPT tubes containing sodium heparin and processed within two hours of collection. PBMCs were isolated from whole blood according to standard protocol provided by product insert. Naive and memory B cells were negatively selected using MACS cell separation reagents to remove non-B as well as pre-existing PC populations. Resulting B cells were plated out under non-differentiating (unsupplemented) or plasma cell differentiating (anti-IgM/anti-CD40/IL-21 supplemented) conditions. After 6.5 days in culture, the antibody dependent cell mediated cytotoxic (ADCC) eliciting potential of the following antibodies was assessed via standard 4 hr ADCC assay protocols: 16C4 afuc (non-fucosylated hIgG1 against CD19), 16C4TM (triple mutant hIgG1 against CD19 modulated for reduced effector activity), hIgG1afuc (non-fucosylated unspecific hIgG1 control) and Rituxan (hIgG1 against CD20). Briefly, spent medium was exchanged and wells were resuspended in appropriate antibody containing media. IL-2 pre-activated KC 1333 NK cells were added at an E:T ratio of 2:1. Cytotoxic efficacy was assessed after 4-6 hours of incubation via staining with Invitrogen fixable live/dead discriminator and B cell markers (CD19, CD20, CD27, and CD38). All samples were stained, fixed and run on an LSR II flow cytometer within 48 hrs.

For primary bone marrow derived PC depletion assays, fresh healthy donor (n=2) human bone marrow was procured from Lonza. Samples were collected in 50 ml conical tubes shipped the same day under refrigerated conditions and processed within two hours of collection. Naive, memory and PC B cells were negatively selected using STEMCELL cell separation reagents to remove non-B cell populations. Resulting B cells were plated out and the ADCC eliciting potential of the test and control antibodies was assessed via standard ADCC assay protocols as described above with the following exceptions: assay incubation time was lengthened to 6 hours and KC1333NK cells were added at an E:T ratio of 1:1.

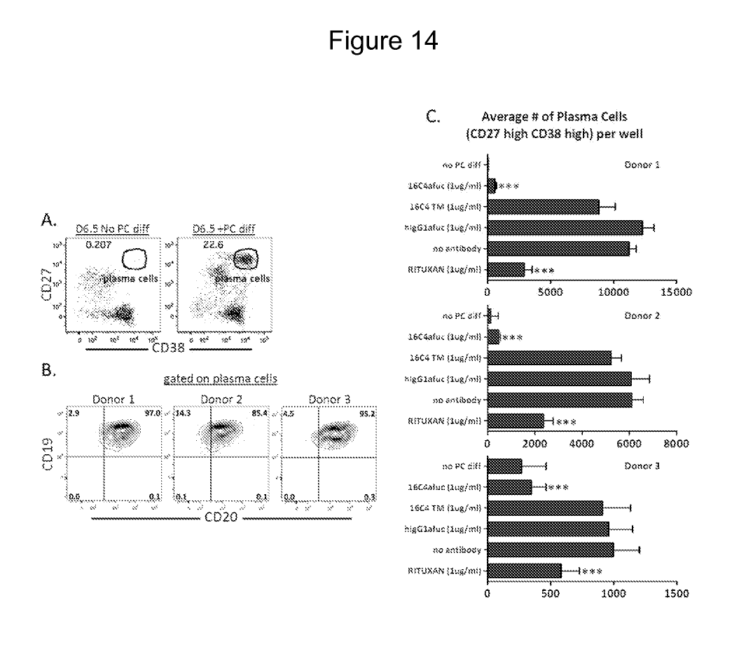

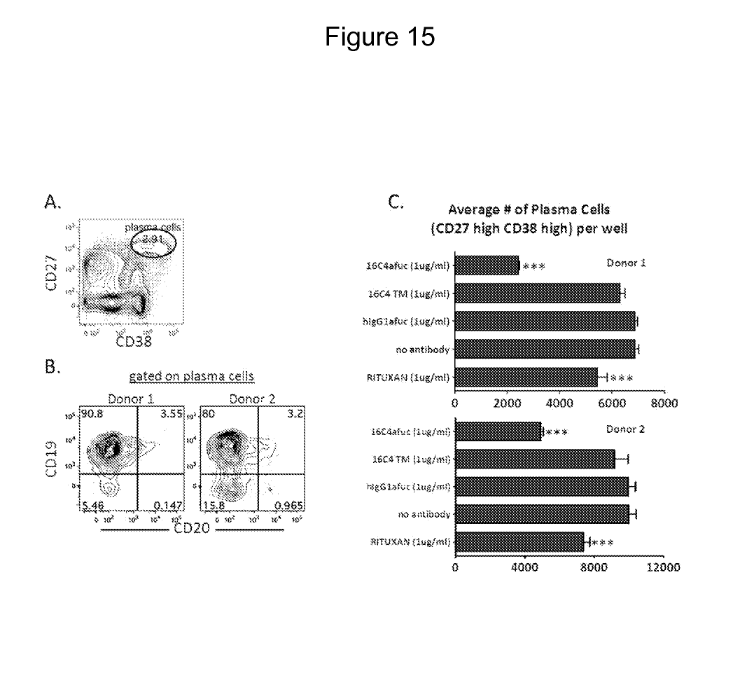

The ADCC data shows 16C4afuc mediated significant depletion of both hPBMC differentiated PC (FIG. 14) as well as freshly isolated PC from human bone marrow (FIG. 15). In both assays, plasma cells were identified flow cytometrically as CD27 high CD38 high lymphocytes within the purified B cell populations (FIGS. 14A and 15B).

As expected, robust differentiation into CD27 high CD38 high PC was dependent upon inclusion of PC differentiation medium (FIG. 14A) and the majority of resultant PC co-expressed CD19 and CD20 (FIG. 15B). Quadrant gates were set using bulk lymphocyte populations (data not shown). In 3 of 3 donors, PC were significantly depleted (p-value ≦0.001) by all tested doses of 16C4afuc. Rituxan dosed at 1 ug/ml also mediated significant depletion of in vitro differentiated PC, though the decrease from no antibody controls was more modest than seed with a similar dose of 16C4afuc. Addition of 16C4 TM and hIgGlafuc control antibodies at 1 ug/ml had minimal affect.

CD19 expression on freshly isolated CD27 high CD38 high PC from human bone marrow was greater than 83% in both donors. CD20 expression was only dimly expressed by fewer than 4% of total PC (FIG. 15B). In 100% of donors, PC were significantly depleted (p-value ≦0.001) by all tested doses of 16C4afuc. Rituxan dosed at 1 ug/ml also mediated significant, though not robust, depletion of freshly isolated bone marrow PC. Addition of 16C4 TM and hIgG1afuc control antibodies at 1 ug/ml had minimal affect.

### Example 14

### FACS Phenotype Analysis of Immune Cells in Whole Blood (WB) and in Cerebrospinal Fluid (CSF) from Multiple Sclerosis (MS) Patients

The presence of plasma cells and plasmablasts in CSF vs. WB of RRMS patients was evaluated and their expression patterns of CD19, in comparison with that of CD20 were examined by FACS.

WB and CSF were collected from RRMS patients. Cells were stained for flow cytometry analysis using a panel of commercially available, fluorescently conjugated antibodies. Cells were washed and then resuspended in PBS/FCS for acquisition. To determine CD19 vs CD20 expression, cells were gated on size, singlets and CD45 (hematopoeitic cell lineage marker).

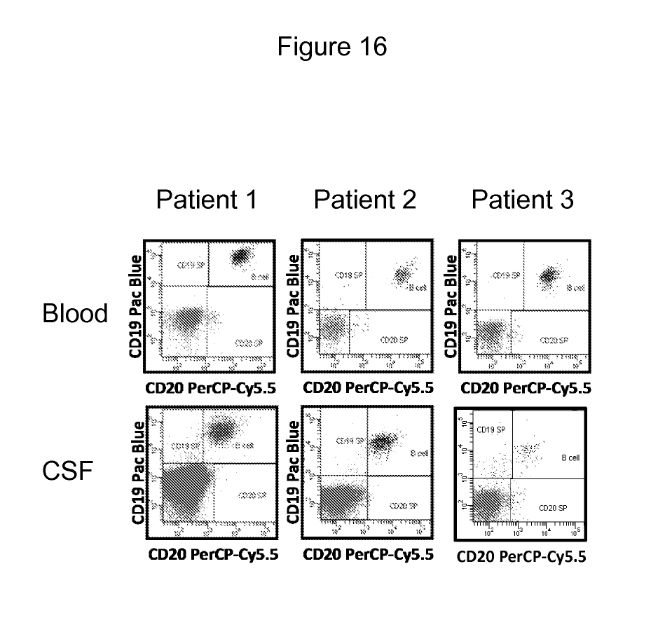

The results showed that CD19+CD20− plasmablasts and plasma cells are enriched in the CSF of RRMS patients. FIG. 16 shows 3 representative flow cytometry plots. CD19 can be detected on surface of B cells from WB and CSF (upper quadrants). While most CD19+ cells also express CD20, a small subset of CD19+ cells in CSF that do not express CD20 (upper left quadrant). These cells are more prevalent in the CSF than WB. CD19+CD20− B cells are reported to be antibody secreting plasmablasts and plasma cells. Other markers on the surface of these cells (CD138, CD27) support this designation (data not shown).

Sequence listing

| | | | |
|---|---|---|---|
| SEQ ID NO: 1 | SSWMN | VH CDR1 | 16C4 |
| SEQ ID NO: 2 | RIYPGDGDTNYNVKFKG | VH CDR2 | 16C4 |
| SEQ ID NO: 3 | SGFITTVRDFDY | VH CDR3 | 16C4 |
| SEQ ID NO: 4 | RASESVDTFGISFMN | VL CDR1 | 16C4 |
| SEQ ID NO: 5 | EASNQGS | VL CDR2 | 16C4 |
| SEQ ID NO: 6 | QQSKEVPFT | VL CDR4 | 16C4 |
| SEQ ID NO: 7 | EVQLVESGGGLVQPGGS | VH | 16C4 |
| | LRLSCAASGFTFSSSWM | | |
| | NWVRQAPGKGLEWVGRI | | |
| | YPGDGDTNYNVKFKGRF | | |
| | TISRDDSKNSLYLQMNS | | |
| | LKTEDTAVYYCARSGFI | | |
| | TTVRDFDYWGQGTLVTV | | |
| | SS | | |
| SEQ ID NO: 8 | EIVLTQSPDFQSVTPKE | VL | 16C4 |
| | KVTITCRASESVDTFGI | | |
| | SFMNWFQQKPDQSPKLL | | |
| | IHEASNQGSGVPSRFSG | | |
| | SGSGTDFTLTINSLEAE | | |
| | DAATYYCQQSKEVPFTF | | |
| | GGGTKVEIK | | |
| SEQ ID NO: 9 | SSWMN | VH CDR1 | 3649 |
| SEQ ID NO: 10 | RIYPGDGDTNYNGKFKG | VH CDR2 | 3649 |
| SEQ ID NO: 11 | SGFITTVLDFDY | VH CDR3 | 3649 |
| SEQ ID NO: 12 | RASESVDTFGISFMN | VL CDR1 | 3649 |
| SEQ ID NO: 13 | AASNQGS | VL CDR2 | 3649 |
| SEQ ID NO: 14 | QQSKEVPFT | VL CDR3 | 3649 |
| SEQ ID NO: 15 | EVQLVESGGGLVQPGGS | VH | 3649 |
| | LRLSCAASGFTFSSSWM | | |
| | NWVRQAPGKGLEWVGRI | | |
| | YPGDGDTNYNGKFKGRF | | |
| | TISRDDSKNSLYLQMNS | | |
| | LKTEDTAVYYCARSGFI | | |
| | TTVLDFDYWGQGTLVTV | | |
| | SS | | |
| SEQ ID NO: 16 | EIVLTQSPDFQSVTPKE | VH | 3649 |
| | KVTITCRASESVDTFGI | | |
| | SFMNWFQQKPDQSPKLL | | |
| | IHAASNQGSGVPSRFSG | | |
| | SGSGTDFTLTINSLEAE | | |
| | DAATYYCQQSKEVPFTF | | |
| | GGGTKVEIK | | |

1. A method of treating multiple sclerosis (MS) disease, comprising administering to a subject in need thereof a therapeutically-effective amount of an antibody, wherein said antibody is a humanized antibody or antigen binding fragment thereof, that binds a CD19 antigen.

2. The method according to claim 1 wherein the multiple sclerosis disease is selected from the group consisting of relapsing-remitting (RR) MS, primary-progressive (PP) MS, secondary-progressive (SP)MS, relapsing-progressive (RP) MS and progressive-relapsing (PR) MS,

3. The method according to claim 2 wherein the multiple sclerosis disease is RRMS.

4. The method according to claim 2 wherein the multiple sclerosis disease is a progressive form of MS selected from PPMS, SPMS, and PR MS

5. The method according to claim 4 wherein the multiple sclerosis disease is PPMS.

6. The method according to claim 4 wherein the multiple sclerosis disease is SPMS

7. The method according to claim 4 wherein the multiple sclerosis disease is PRMS

8. The method of any one of claims 1 to 7, wherein the antibody comprises a VH and a VL, wherein the VH comprises a VH CDR1 having at least 95% identity to the amino acid sequence of SEQ ID NO: 1, a VH CDR2 having at least 95% identity to the amino acid of SEQ ID NO: 2 and VH CDR3 having at least 95% identity to the amino acid sequence of SEQ ID NO: 3.

9. The method of claim 8, wherein the antibody comprises a VH and a VL, wherein the VH comprises a VH CDR1 having an amino acid sequence of SEQ ID NO: 1, a VH CDR2 having an amino acid of SEQ ID NO: 2 and VH CDR3 having an amino acid sequence of SEQ ID NO: 3

10. The method of any one of claims 1 to 9, wherein the antibody comprises a VH and a VL, wherein the VL comprises a VL CDR1 having at least 95% identity to the amino acid sequence of SEQ ID NO: 4, a VL CDR2 having at least 95% identity to the amino acid of SEQ ID NO: 5 and VL CDR3 having at least 95% identity to the amino acid sequence of SEQ ID NO: 6.

11. The method of claim 10, wherein the antibody comprises a VH and a VL, wherein the VL comprises a VL CDR1 having an amino acid sequence of SEQ ID NO: 4, a VL CDR2 having an amino acid of SEQ ID NO: 5 and VL CDR3 having an amino acid sequence of SEQ ID NO: 6

12. The method of any of claims 1-11, wherein the VH comprises an amino acid sequence having at least 95% identity to SEQ ID NO: 7.

13. The method of any of claims 1-11, wherein the VH comprises an amino acid sequence having of SEQ ID NO: 7

14. The method of any of claims 1-13, wherein the VL comprises an amino acid sequence having at least 95% identity to SEQ ID NO: 8.

15. The method of any of claims 1-14, wherein the VL comprises an amino acid sequence of SEQ ID NO: 8

16. The method of any of claims 1-15, wherein the antibody comprises an Fc variant, wherein the Fc variant has an altered affinity for one or more Fc ligands selected from the group consisting of: C1q, FcγRI, FcγRIIA, FcγRIIB and FcγRIIIA.

17. The method of claim 16, wherein the Fc variant has an affinity for the Fc receptor FcγRIIIA that is at least about 5 fold lower than that of a comparable molecule, and wherein said Fc variant has an affinity for the Fc receptor FcγRIIB that is within about 2 fold of that of a corresponding non-variant Fc molecule.

18. The method of any of claims 1-17, wherein the antibody has an enhanced ADCC activity.

19. The method of any of claims 1-18, wherein the method comprises depletion of B cells selected from the group consisting of: circulating B cells, blood B cells, splenic B cells, marginal zone B cells, follicular B cells, peritoneal B cells and bone marrow B cells.

20. The method of any of claims 1-19, wherein the method comprises depletion of B cells selected from the group consisting of: progenitor B cells, early pro-B cells, late pro-B cells, large-pre-B cells, small pre-B cells, immature B cells, mature B cells, antigen stimulated B cells and plasma cells.

21. The method of claim 9 or 10, wherein the depletion reduces B cell levels by at least about 20%, at least about 30%, at least about 40%, at least about 50%, at least about 60%, at least about 70%, at least about 80%, at least about 90%, at least about 95%, or about 100%.

22. The method of any of claims 9-11, wherein the depletion persists for a time period selected from the group consisting of: at least 1 week, at least 2 weeks, at least 3 weeks, at least 4 weeks, at least 5 weeks, at least 6 weeks, at least 7 weeks, at least 8 weeks, at least 3 months, at least 4 months, at least 5 months, at least 6 months, at least 7 months, at least 8 months, at least 9 months, at least 10 months, at least 11 months or at least 12 months.

23. The method of any of claims 1-12, wherein the antibody is conjugated to a cytotoxic agent.

24. The method of any of claims 1-23, wherein the antibody is co-administered with an anti-CD20, anti-CD52, or anti-CD22 antibody.

25. The method of any of claims 1-24, wherein the antibody is co-administered with an interferon-beta, Copaxone™, corticosteroids, cyclosporine, calcineurin inhibitors, azathioprine, Rapamune™, Cellcept™, methotrexate or mitoxantrone

26. A method of treating multiple sclerosis in a human, comprising administering to a patient in need thereof a composition comprising a plurality of monoclonal antibodies that bind a CD 19 antigen, wherein 80-100% of the antibodies are afucosylated.

27. The method of claim 16, wherein the antibody is as defined in any one of claims 8 to 18.
